(19) United States
(12) Patent Application Publication
(10) Pub. No.: US 2024/0269484 A1
(43) Pub. Date: Aug. 15, 2024

(54) SYSTEMS AND METHODS FOR PATIENT TRACKING DURING RADIATION THERAPY

(71) Applicant: The Brigham and Women's Hospital, Inc., Boston, MA (US)

(72) Inventors: Ross Berbeco, Cambridge, MA (US); Matthew Jacobson, Natick, MA (US); Thomas Harris, Brookline, MA (US)

(21) Appl. No.: 18/563,276

(22) PCT Filed: May 26, 2022
(86) PCT No.: PCT/US22/31017

§ 371 Date: Nov. 21, 2023

Related U.S. Application Data
(60) Provisional application No. 63/193,448, filed on May 26, 2021.

(51) Int. Cl. A61N 5/10

(52) U.S. Cl. CPC A61N 5/1049; A61N 5/1037; A61N 5/1071; A61N 5/1081; A61N 2005/1054; A61N 2005/1061

(57) ABSTRACT

A method is provided for delivering radiation therapy to a patient. The method can include receiving a radiation treatment plan for the patient, receiving first imaging data, the first imaging data can be a dual-energy imaging acquisition, delivering a radiation therapy beam to a patient, receiving, from a portal imaging system, second imaging data, the portal imaging system can sense the radiation therapy beam to acquire the second imaging data, and compensating for movement of the patient, based on the first imaging data and the second imaging data.

### CROSS-REFERENCE TO RELATED APPLICATIONS

This application claims priority to U.S. Patent Application No. 63/193,448 filed May 26, 2021, and entitled, “Systems and Methods for Patient Tracking During Radiation Therapy,” which is hereby incorporated by reference in its entirety.

### STATEMENT REGARDING FEDERALLY SPONSORED RESEARCH

This invention was made with government support under R01-CA-188446, awarded by the National Institutes of Health. The government has certain rights in the invention.

### BACKGROUND

During external beam radiotherapy (“EBRT”) procedures, clinicians sometimes request cone-beam computed tomography (“CBCT”) images (e.g., kilo-voltage CBCT images) to be acquired to verify patient positioning. However, repeated checks of patient positioning with CBCT can be slow (e.g., lengthening the treatment time) and can expose the patient to extra radiation (e.g., from the CBCT imaging system). Thus, it would be desirable to provide improved systems and methods for patient tracking during radiation therapy.

### SUMMARY OF THE DISCLOSURE

Some non-limiting examples of the disclosure provide a system for delivering radiotherapy to a patient. The system can include a radiotherapy system having a radiation source, an imaging system, a portal imaging system configured to sense radiation emitted from the radiation source of the radiotherapy system, and a computing device. The computing device can be configured to receive a radiation treatment plan for the patient, the radiation treatment plan can define a first beam path to a target site of the patient and a second beam path to the target site of the patient, receive, from the imaging system, first imaging data, the first imaging data can include a first set of imaging data acquired at a first energy level, and a second set of imaging data acquired at a second energy level different from the first energy level, the first and second sets of imaging data can each include a region of interest of the patient that includes the target site, cause the radiation source to emit a radiation therapy beam along the first beam path to the target site of the patient, receive, from the portal imaging system, second imaging data acquired during emission of the radiation therapy beam along the first beam path, determine movement of the patient based on the first imaging data and the second imaging data, and adjust the radiation treatment plan to compensate for the determined movement of the patient.

In some non-limiting examples, a computing device can be configured to generate, using first and second sets of CBCT imaging data, a three-dimensional (3D) volume of a region of interest of a patient at an energy of a radiation therapy beam, receive or generate, using the x-ray imaging data, a plurality of BEV portal images, perform a 3D-2D registration between the 3D volume and the portal imaging system using the plurality of BEV images, and determine movement of the patient, based on the 3D-2D registration.

In some non-limiting examples, a computing device can be configured to generate, using a 3D volume, a plurality of two-dimensional (2D) digital radiograph images, and perform a 3D-2D registration using the 2D digital radiograph images and a plurality of BEV portal images.

In some non-limiting examples, a 3D-2D registration can be a mono-modality registration.

In some non-limiting examples, a radiation treatment plan can define a first sweep path of a radiation source that includes a first beam path, and a second sweep path of the radiation source that includes a second beam path. The first sweep can be different than the second sweep.

In some non-limiting examples, a first sweep path can be a first arc path to be followed by a radiation source. A second sweep path can be a second arc path to be followed by the radiation source.

In some non-limiting examples, a computing device can be configured to generate, using first imaging data, a three-dimensional (3D) volume of a region of interest of a patient, perform a 3D-2D registration between the 3D volume and the portal imaging system using the x-ray imaging data, and determine movement of the patient, based on the 3D-2D registration.

In some non-limiting examples, x-ray imaging data can be acquired by a portal imaging system during emission of another radiation therapy beam along a second sweep path.

In some non-limiting examples, adjust a radiation treatment plan to compensate for determined movement can include a computing device being configured to change a property of a next radiation therapy beam to be directed at the patient, according to the radiation treatment plan and based on the determined movement. The next radiation therapy beam can correspond to a next beam path to be followed by the next radiation therapy beam. In some cases, adjust the radiation treatment plan to compensate for the determined movement can include the computing device being configured to change an order of application of any of the remaining radiation treatment beams to be delivered to the patient according to the radiation treatment plan and based on the determined movement, each beam path of the radiation treatment plan can correspond with each radiation treatment beam, remove a radiation treatment beam that was to be directed at a patient along a beam path from the radiation treatment plan, based on the determined movement, or move the coordinates of a reference point defined previously in the radiation treatment plan, based on the determined movement.

In some non-limiting examples, a computing device can be configured to change a property of a next radiation therapy beam to be directed at a patient, according to a radiation treatment plan and based on determined movement of the patient. The next radiation therapy beam can correspond to a next beam path to be followed by the next radiation therapy beam. The property of the next radiation therapy beam can include at least one of a duration of application of the next radiation therapy beam, a radiation dose provided by the next radiation therapy beam, an energy level of the next radiation therapy beam, a position of the beam path to be followed by the next radiation therapy beam, or an orientation of the beam path to be followed by the next radiation therapy beam.

In some non-limiting examples, determining an adjustment to a radiation treatment plan to compensate for determined movement of a patient can include a computing device being configured to move at least one of a patient or a radiation source to compensate for the determined movement.

In some non-limiting examples, a computing device can be configured to generate, using first imaging data, a first 3D volume of the patient; receive or generate, using fourth imaging data, a second 3D volume of the patient, register the first 3D volume with the second 3D volume, determine another movement of the patient based on the registration between the 3D volumes, before causing the radiation source to emit the radiation therapy beam, and determine another adjustment to a radiation treatment plan to compensate for the another determined movement of the patient.

In some non-limiting examples, a first set of CBCT imaging data acquired at a first energy level can include an imaging system emitting a first radiation imaging beam having a first radiation energy spectrum towards the patient and a detector array of the imaging system. The first energy level being a peak in the first radiation energy spectrum. A second set of CBCT imaging data acquired at a second energy level can include the imaging system emitting a second radiation imaging beam having a second radiation energy spectrum towards the patient and the detector array of the imaging system. The second energy level can be a peak in the second radiation energy spectrum.

Some non-limiting examples of the disclosure provide a computer-implemented method for tracking a patient. A method can include receiving, using one or more computing devices, a radiation treatment plan. The radiation treatment plan can define a first beam path to a target site of the patient and a second beam path to the target site of the patient. The method can include receiving, using the one or more computing devices, first imaging data. The first imaging data can include a first set of CBCT imaging data of a region of interest of the patient and acquired at a first energy level. The region of interest can include the target site. The first imaging data can include a second set of CBCT imaging data of the region of interest and acquired at a second energy level different from the first energy level. The method can include receiving, using the one or more computing devices, x-ray imaging data acquired from a portal imaging system during emission of a radiation therapy beam along the first beam path, determining, using the one or more computing devices, movement of the patient based on the first imaging data and the portal imaging data, and updating, using the one or more computing devices, a position of the patient, based on the movement of the patient.

In some non-limiting examples, a method can include moving, using one or more computing devices, a patient or a radiation source to compensate for determined movement of the patient.

In some non-limiting examples, updating a position of a patient based on movement of the patient can include changing, using one or more computing devices, a property of a next radiation therapy beam to be directed at the patient, according to a radiation treatment plan and based on the determined movement. The next radiation therapy beam can correspond to the next beam path to be followed by the next radiation therapy beam. In some non-limiting examples, updating the position of the patient based on movement of the patient can include changing, using the one or more computing devices, an order of application of any of the remaining radiation treatment beams to be delivered to the patient according to the radiation treatment plan and based on the determined movement, each beam path of the radiation treatment plan can correspond with each radiation treatment beam, removing, using the one or more computing devices, a radiation treatment beam that was to be directed at a patient along a beam path from the radiation treatment plan, based on the determined movement; or moving, using the one or more computing devices, the coordinates of a reference point defined previously in the radiation treatment plan, based on the determined movement.

In some non-limiting examples, a method can include generating, using one or more computing devices and CBCT imaging data, a three-dimensional (3D) volume of a region of interest of a patient, performing, using the one or more computing devices and the x-ray imaging data, a 3D-2D registration between the 3D volume and the portal imaging system, and determining, using the one or more computing devices, movement of the patient, based on the 3D-2D registration.

Some non-limiting examples of the discourse a method for delivering radiation therapy to a patient. The method can include receiving a radiation treatment plan for the patient, receiving first imaging data, the first imaging data can be a dual-energy imaging acquisition, delivering a radiation therapy beam to a patient, receiving, from a portal imaging system, second imaging data, the portal imaging system can sense the radiation therapy beam to acquire the second imaging data, and compensating for movement of the patient, based on the first imaging data and the second imaging data.

In some non-limiting examples, a method can include generating, using first imaging data, a three-dimensional (3D) volume of a region of interest of a patient, performing, using the second imaging data, a 3D-2D registration between the 3D volume and the portal imaging system, and determining movement of the patient, based on the 3D-2D registration.

Some non-limiting examples of the disclosure provide a system for delivering radiotherapy to a patient. The system can include a radiation therapy source configured to move to different positions about the patient, and an imaging system including one or more radiation imaging sources and one or more detector arrays. The one or more radiation imaging sources can be configured to emit different radiation imaging beams having different energies towards the patient and the one or more detector arrays to acquire imaging data of the patient at different energies. The system can include a portal imaging device configured to sense radiation emitted from the one or more radiation sources to receive imaging data therefrom, and a computing device. The computing device can be configured to receive, from the imaging system, first imaging data of a region of interest of the patient that includes a target site receive, from the imaging system, second imaging data of the region of interest of the patient, generate a 3D volume of the region of interest of the patient using the first imaging data and the second imaging data, cause the radiation therapy source to emit a first radiation therapy beam at the target site of the patient according to a radiation treatment plan, receive, from the portal imaging device, one or more beam eye view (“BEV”) images from the interaction between the first radiation therapy beam and the portal imaging device, after the first radiation therapy beam has passed through the patient, determine a movement of the patient, based on the 3D volume and the one or more BEV images, and update or change the radiation treatment plan, based on the determined movement of the patient.

In some non-limiting examples, first imaging data can be acquired with one or more radiation sources of an imaging system operating at a first energy level. Second imaging data can be acquired with the one or more radiation sources of the imaging system operating at a second energy level different from the first energy level.

In some non-limiting examples, a first energy level can be a first peak energy of radiation emitted by the one or more radiation sources. A second energy level can be a second peak energy of radiation emitted by the one or more radiation sources. The first peak energy can be different than the second peak energy.

In some non-limiting examples, a first energy level can correspond to a first tissue type being visible in a first imaging data. A second energy level can correspond to a second tissue type different from the first tissue type being visible in second imaging data.

In some non-limiting examples, first tissue type can be a water-based tissue type, and second tissue type can be bone. In some cases, the water-based tissue has a greater than 31 percent by weight amount of water.

In some non-limiting examples, receiving first imaging data of a region of interest can include causing one or more radiation sources of an imaging system to emit a first radiation imaging beam towards the region of interest of a patient, the first radiation imaging beam can have a first radiation energy spectrum, the first energy level can be a peak in the first radiation energy spectrum, and receiving, from one or more detector arrays of the imaging system, the first imaging data from the interaction between the first radiation imaging beam and the one or more detector arrays. In some non-limiting examples, receiving first imaging data of the region of interest can include causing the one or more radiation sources of the imaging system to emit a second radiation imaging beam towards the region of interest of the patient, the second radiation imaging beam can have a second radiation energy spectrum, the second energy level can be a peak in the second radiation energy spectrum, and receiving, from the one or more detector arrays of the imaging system, the second imaging data from the interaction between the first radiation imaging beam and the one or more detector arrays.

In some non-limiting examples, determining a movement of a patient based on a 3D volume and one or more BEV images can include performing a 3D-2D registration between the 3D volume and the portal imaging device using the 3D volume and the one or more BEV images, and determining the movement of the patient, based on the 3D-2D registration.

In some non-limiting examples, performing a 3D-2D registration between a 3D volume and a portal imaging device can include generating, using the 3D volume, one or more two-dimensional (2D) digital radiograph images, each 2D digital radiograph image can correspond to a position of a radiation therapy source relative to a patient during emission of a first radiation therapy beam, and perform the 3D-2D registration by using the one or more 2D digital radiograph images and the one or more BEV images.

In some non-limiting examples, a portal imaging device can include a plurality of sensing layers. Each of the one or more BEV images can be generated using the plurality of sensing layers.

In some non-limiting examples, causing a radiation therapy source to emit a first radiation therapy beam at a target site of a patient according to a radiation treatment plan can include causing the radiation therapy source to emit the first radiation beam at the target site while moving the radiation therapy source along an arc.

In some non-limiting examples, a computing device can be configured to after updating or changing a radiation treatment plan, moving a radiation therapy source to a position relative to a patient, and with the radiation therapy source at least at the position, causing the radiation therapy source to emit a second radiation therapy beam at a target site of the patient according to the radiation treatment plan.

In some non-limiting examples, one or more BEV images can be one or more first BEV images. A computing device can be configured to receive, from a portal imaging device, one or more second BEV images from the interaction between a second radiation therapy beam and the portal imaging device, after the second radiation therapy beam has passed through the patient, determine a further movement of the patient, based on the 3D volume and the one or more second BEV images, and further update or change the radiation treatment plan, based on the determined further movement of the patient.

In some non-limiting examples, a method can include after further updating or changing a radiation treatment plan, moving a radiation therapy source to another position relative to a patient, and with the radiation therapy source at least at the another position, causing the radiation therapy source to emit a third radiation therapy beam at a target site of the patient according to the radiation treatment plan.

In some non-limiting examples, a computing device can be configured to perform a first 3D-2D registration between a 3D volume and a portal imaging device using the 3D volume and one or more first BEV images, determine movement of the patient, based on the first 3D-2D registration, perform a second 3D-2D registration between the 3D volume and the portal imaging device using the 3D volume and one or more second BEV images; and determine further movement of the patient, based on the second 3D-2D registration.

In some non-limiting examples, updating or changing a radiation treatment plan can include adjusting a reference point of the radiation treatment plan, based on the determined movement. Each radiation therapy beam of the radiation treatment plan can be defined relative to the reference point. In some non-limiting examples, updating or changing the radiation treatment plan can include adjusting one or more other radiation therapy beams of the radiation treatment plan other than a first radiation therapy beam.

Some non-limiting examples of the disclosure provide a method of irradiating a patient according to a radiation treatment plan. The method can include receiving a 3D volume of a region of interest of the patient that includes a target site, the 3D volume having been generated by irradiating the patient with radiation imaging beams having different peak energies, causing a radiation therapy source to emit a radiation therapy beam at the target site of the patient according to the radiation treatment plan receiving, from a portal imaging device, one or more beam eye view (“BEV”) images from the interaction between the radiation therapy beam and the portal imaging device, after the radiation therapy beam has passed through the patient determining a movement of the patient, based on the 3D volume and the one or more BEV images, and compensating for the movement of the patient to more accurately deliver one or more additional radiation therapy beams to the target site of the region of interest of the patient, the one or more additional radiation therapy beams being according to the radiation treatment plan.

In some non-limiting examples, a radiation therapy source can emit a radiation therapy beam while the radiation therapy source moves about the patient along a first sweep path. A method can include generating, using a 3D volume, one or more two-dimensional (2D) digital radiograph images, each 2D digital radiograph image can correspond to one or more different positions of the radiation therapy source relative to the patient during emission of the radiation therapy beam, performing a 3D-2D registration between the 3D volume and a portal imaging device using the one or more 2D digital radiograph images and one or more BEV images; and determining movement of the patient, based on the 3D-2D registration.

In some non-limiting examples, a radiation therapy beam can be a first radiation therapy beam. A method can include after compensating for a movement of a patient, moving a radiation therapy source to a position relative to the patient, and with the radiation therapy source at least at the position, causing the radiation therapy source to emit a second radiation therapy beam at a target site of the patient according to a radiation treatment plan.

In some non-limiting examples, a radiation therapy source can emit a second radiation therapy beam while the radiation therapy source moves about the patient along a second sweep path different from a first sweep path.

In some non-limiting examples, a first sweep path can be a first arc about a patient, and a second sweep path can be a second arc about the patient.

Some non-limiting examples of the disclosure provide a computer-implemented method for tracking a patient. The method can include receiving, using one or more computing devices, a radiation treatment plan, the radiation treatment plan defining a first beam path to a target site of the patient and a second beam path to the target site of the patient, and receiving, using the one or more computing devices, first imaging data. The first imaging data can include a first set of imaging data of a region of interest of the patient and acquired at a first energy level, the region of interest including the target site, and a second set of imaging data of the region of interest and acquired at a second energy level different from the first energy level, the region of interest including the target site. The method can include receiving, using the one or more computing devices, second imaging data acquired from a portal imaging system during emission of a radiation therapy beam along the first beam path, determining, using the one or more computing devices, movement of the patient based on the first imaging data and the second imaging data, and updating, using the one or more computing devices, a position of the patient, based on the movement of the patient.

Some non-limiting examples of the disclosure provide a method for delivering radiation therapy to a patient. The method can include receiving a radiation treatment plan for the patient, receiving first imaging data, the first imaging data being a dual-energy imaging acquisition, delivering a radiation therapy beam to a patient, receiving, from a portal imaging system, second imaging data, the portal imaging system sensing the radiation therapy beam to acquire the second imaging data, and compensating for movement of the patient, based on the first imaging data and the second imaging data.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a schematic illustration of a system for delivering radiotherapy to a patient.

FIG. 2A shows an isometric view of an imaging system.

FIG. 2B shows a schematic illustration of the imaging system of FIG. 2A.

FIG. 3 shows a portion of a flowchart of a process for delivering radiation therapy to a patient, which can include a process of tracking a patient (e.g., during radiotherapy).

FIG. 4 shows a portion of the flowchart of the process for delivering radiation therapy to the patient.

FIG. 5 shows a schematic illustration of a process that was used to track movement of digitally constructed images.

FIG. 6A shows a digital thorax image that was generated according to a simulated first energy level.

FIG. 6B shows a digital thorax image that was generated at a simulated second energy level.

FIG. 6C shows images from a simulated treatment beam showing tracking of the spine between images.

FIG. 7 shows an experimental setup of an imaging detector for a portal imaging system.

FIG. 8A shows a graph of the shift estimation error verses the number of views for a true shift in 1 cm for the digital thorax study.

FIG. 8B shows a graph of the estimated shift magnitude verses true shift magnitudes for the digital thorax study.

FIG. 9A shows a digital abdomen image that was generated according to a simulated first energy level.

FIG. 9B shows a digital abdomen image that was generated at a simulated second energy level.

FIG. 9C shows images from a simulated treatment beam showing tracking of the spine between images.

FIG. 10A shows a graph of the shift estimation error verses the number of views for a true shift in 1 cm for the digital abdomen study.

FIG. 10B shows a graph of the estimated shift magnitude verses true shift magnitudes for the digital abdomen study.

### DETAILED DESCRIPTION OF THE PRESENT DISCLOSURE

Sometimes during radiotherapy procedures (e.g., EBRT), clinicians request that kilo-voltage CBCT (“kV CBCT”) images are acquired, which can be used to ensure that the patient is properly positioned. For example, the radiation treatment plan for the patient can include a number of different beam paths (e.g., each corresponding to a different external radiation beam). In particular, the radiation treatment plan can include a number of different treatment arcs, with each treatment arc defining a sweep of the radiation beam along an arc. However, between different arcs (or other positions of the radiation source), the patient could move (e.g., in a relatively small manner), and thus the subsequent radiation beam (e.g., along the corresponding arc) could be delivered to an unintended location of the patient (e.g., healthy tissue rather than the target tissue including diseased tissue). Thus, in some cases, between each treatment arc, the clinician can request a CBCT to ensure that the patient is positioned according to the radiation treatment plan (e.g., an expected position). If the clinician is not satisfied with the positioning of the patient, the patient support position is adjusted and another CBCT scan is taken to verify the adjusted position. This process is repeated iteratively until the clinician is satisfied. While this CBCT process can ensure that the patient is positioned properly, there are downsides to this process. For example, these repeated CBCT imaging acquisitions between treatment arcs can significantly extend the total length of the radiotherapy treatment, thereby decreasing the efficiency of the radiotherapy system. In other words, the current radiotherapy session can be decreased in accuracy and efficiency (e.g., due to patient discomfort and thus undesirable patient movement during treatment). In addition, due to the decreased efficiency of the current radiotherapy session (and others), the radiotherapy system is not optimized to treat the maximum number of patients that require radiation therapy treatment within a period of time (e.g., a week). As another example, aside from the inefficiencies of the CBCT imaging acquisitions, these acquisitions undesirably expose the patient to additional radiation doses.

Some non-limiting examples of the disclosure provide advantages to address these issues (and others) by providing improved systems and methods for patient tracking during radiation therapy. For example, some non-limiting examples of the disclosure provide a system for delivering radiotherapy to a patient, which can include a radiotherapy system having a radiation source, an imaging system, and a portal imaging system. The imaging system is configured to acquire a dual-energy imaging acquisition (or in other words a spectral imaging acquisition), which can include two sets of imaging data with each set acquired at a different energy level. For example, the imaging system can have two radiation sources with each radiation source operating at a different energy level, while in other cases, the imaging system can have a radiation source that operates at two different energy levels. Regardless, before the start of treatment, a first set of CBCT imaging data is acquired while the radiation source operates at a first energy level, and a second set of CBCT imaging data is acquired while the radiation source operates at a second energy level different from the first energy level. With these techniques (e.g., as used in the field of dual energy CT (“DECT”)), the two imaging data sets are used to generate a 3D CBCT volume at a third energy level (e.g., the energy of the treatment beam), which can be different than (e.g., greater than) the energy levels used to acquire the first and second sets of CBCT imaging data. This 3D CBCT image volume can be used to predict what the radiotherapy portal imager would measure for various hypothetical positions of the patient (and orientations of the radiotherapy beam).

As the first radiation therapy beam is delivered, the portal imaging system acquires 2D portal imaging data (e.g., the portal imaging system sensing radiation emitted from the first radiation therapy beam). Then, once the first radiation therapy beam has been delivered (e.g., after the first radiation therapy beam sweeps an arc), a 3D-2D registration is performed between the extrapolated 3D CBCT volume and the actual 2D portal imaging system measurements. Because the 3D CBCT image volume is extrapolated to the same (or similar) energy level as the treatment beam, the registration process can form predictions of the 2D portal imaging data that highly resemble the actual portal imager measurements (because image features better resemble each other at similar energy levels), and a mono-modality registration technique can therefore be used. In this way, with these systems registered, two-dimensional (“2D”) images from the portal imaging system (e.g., beam eye view (“BEV”) images) can be used to track the position of the patient. In particular, the 2D images are advantageously referenced to the 3D volume and thus 3D features of the patient can be tracked using only the 2D images from the portal imaging system. This provides at least two important advantages. First, because the portal imaging system is used to track the patient, the trial-and-error CBCT acquisitions can be eliminated from the patient re-positioning process thereby increasing clinical efficiency and preventing the patient from receiving additional radiation. Second, because the tracking process relies on mono-modality registration, faster and more accurate registration algorithms can be used, as compared to alternative multi-modality registration approaches.

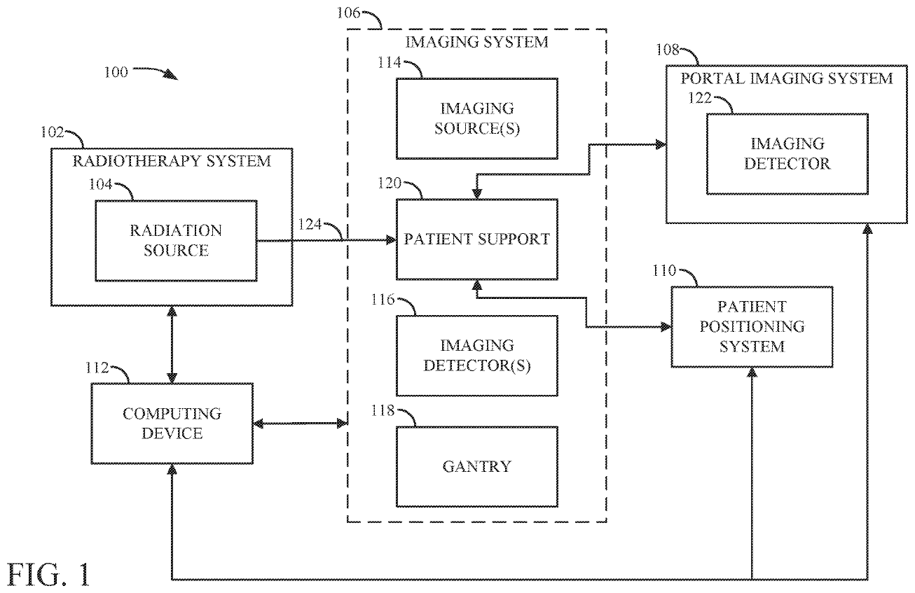

FIG. 1 shows a schematic illustration of a system 100 for delivering radiotherapy to a patient. The system 100 can include a radiotherapy system 102 having a radiation source 104, an imaging system 106, a portal imaging system 108, a patient positioning system 110, and a computing device 112. In some cases, the radiation source 104 can include an X-ray beam forming assembly configured to emit X-rays, or in other cases, the radiation source 104 can be a linear accelerator (“LINAC”) or other particle accelerator configured to accelerate particles (e.g., electrons, protons, etc.), such as, a cyclotron (e.g., a synchrotron). Although not shown, the radiotherapy system 102 can include a gantry that supports the radiation source 104. The gantry can be moveable so that when the gantry moves, the radiation source 104 moves with the gantry. In some cases, the gantry can be a cylinder gantry, a ring gantry, a C-arm gantry, etc. In other configurations, the radiotherapy system 102 can include a robotic arm, where the radiation source 104 is coupled to the robotic arm (e.g., a distal end of the robotic arm, so that the radiation source 104 is the end effector of the robotic arm). As shown in FIG. 1, the computing device 112 is in communication with the radiotherapy system 102, and thus the computing device 112 can control aspects of the radiotherapy system 102. For example, the computing device 112 can selectively control the radiation source (e.g., by turning on the radiation source 104 to emit a radiation beam or by turning the radiation source 104 off to stop the radiation source 104 from emitting the radiation beam). As another example, the computing device 112 can move the position of the radiation source 104, such as by rotating the gantry and thus the radiation source 104, rotating the radiation source 104 about the gantry, moving a robotic arm and thus the radiation source 104, etc. Thus, regardless of the configuration, the radiotherapy system 102 (e.g., via instruction by the computing device 112), can cause the radiation source 104 to emit radiation therapy beams that each follow different beam paths, which can be according to a radiation treatment plan for the patient.

In some non-limiting examples, the radiation treatment plan for the patient can include a plurality of beam paths for different (or the same) radiation therapy beams to be emitted along, with each beam path being different from each other. In some cases, the radiation treatment plan can include a plurality of sweeps of the radiation therapy beam, each of which can also be different from each other. For example, as a radiation therapy beam is being emitted, the radiation therapy beam moves along a path (e.g., swept along a path). In some cases, the path can be continuous (or non-continuous), a curved path (e.g., an arc) having various lengths and curvatures, or a straight path (e.g., a non-curved path) having various lengths and slopes relative to the patient.

In some non-limiting examples, the radiation source 104 can include an adjustable collimator (e.g., a multi-leaf collimator) that can adjust its radiation attenuation profile (e.g., how a radiation beam is attenuated as it passes through the adjustable collimator). For example, such as when the adjustable collimator is a multi-leaf collimator, the collective positioning of the leaves of the multi-leaf collimator can define the radiation attenuation profile (e.g., among other features, such as the thickness of each leaf, etc.). In some configurations, the radiotherapy system 102 can adjust the attenuation profile of the adjustable collimator, based on a radiation beam that is to be delivered to a patient according to a radiation treatment plan for the patient. In this way, different radiation therapy beams with different characteristics (e.g., size, shape, attenuation profile, etc.). can be delivered to the patient during the radiotherapy treatment. In some non-limiting examples, the radiation source 104 is configured to operate at different energies to deliver radiation therapy beams with different energy levels. For example, the radiation source 104 can emit a radiation therapy beam having an energy that is substantially (i.e., deviating by less than 20% from) 500 keV, and can have a peak energy of 6 mega volts peak (“MVp”).

In some non-limiting examples, the system 100 can include an imaging system 106. The imaging system 106 is configured to acquire imaging data of a region of interest of a patient (e.g., a tumor site and tissue surrounding the tumor site). In some cases, this imaging data can be used to generate a three-dimensional (“3D”) volume of the region of interest of the patient, which can be helpful in planning purposes. For example, with the patient situated on a patient support 120 of the patient positioning system 110, the imaging system 106 can acquire imaging data of the region of interest of the patient. In some cases, this imaging data can be used to generate a radiation treatment plan for the patient. In other cases, such as with the radiation treatment plan already having been created (e.g., including based on other imaging data from the imaging system 106 or another imaging system), the imaging data can be used to verify the position of the patient (e.g., relative to the patient support 120). For example, the generated 3D volume of the region of interest of the patient can be compared to a previously generated 3D volume of the patient (e.g., by comparing common features between the 3D volumes), and this comparison can be used to determine whether or not the patient has moved. If the patient has moved, the radiation treatment plan can be adjusted, which can include adjusting the position of some (or all) of the beam paths of the radiation treatment plan. In some non-limiting examples, the imaging system 106 can be a CT imaging system (e.g., a CBCT imaging system).

In some non-limiting examples, the imaging system 106 is configured to acquire imaging data that includes a first set of imaging data acquired at a first energy level, and a second set of imaging data acquired at a second energy level different from the first energy level. In other words, the imaging system 106 can be configured to acquire imaging data that is a dual-energy imaging acquisition. Thus, the imaging system 106 can be a dual-energy imaging system (e.g., a dual-energy x-ray imaging system), a multi-energy imaging system (e.g., a multi-energy x-ray imaging system), etc. The imaging system 106 that can include its own computing device (or a different computing device) can combine the first and second sets of imaging data, and can generate a 3D volume of the patient from the combined imaging data. In some cases, this can include a computing device constructing an image from the first set of imaging data acquired at an orientation relative to the patient, constructing an image from the second set of imaging data acquired at the same orientation relative to the patient, combining the images (e.g., by superimposing the images, averaging the images, etc.) and generating a 3D volume of the patient from the combined images. In some cases, this process can be used for a plurality of pairs of constructed images, with each pair having one image from the first imaging data and another image from the second imaging data both of which are acquired for the same portion of the patient (e.g., the same projection of the patient).

In some non-limiting examples, the imaging system 106 can include an imaging source 114, an imaging detector 116 that can be aligned with the imaging source 116, and a gantry 118 that can support one or both of the imaging source 114 or the imaging detector 116. The imaging source 114 can be configured to emit a radiation imaging beam towards the patient, and the imaging detector 116 can be configured to sense the radiation that passes through the patient. The imaging source 114 can be structured in a similar manner as the radiation source 104. For example, the imaging source 114 can include an X-ray beam forming assembly configured to emit X-rays (e.g., an X-ray beam) toward the patient. In some configurations, the imaging source 114 is configured to acquire CBCT data sets at multiple X-ray energies (e.g., two energies, three energies, four energies, with each energy being different), which can be in a single gantry rotation. This may be accomplished, for example, using a pair of x-ray source-detector subsystems each running at a separate energy. Alternatively, it may be accomplished with a single source-detector subsystem which rapidly switches between source energies during gantry rotation. In some non-limiting examples, the computing device 112 (or a different computing device) can cause the imaging source 114 to switch between single energy and multi-energy modes (e.g., a dual energy mode) of operation.

In configurations in which the imaging system 106 includes multiple imaging sources, each imaging source 114 can be positioned at known distances from each other on the imaging system 106. For example, each imaging source 114 can be coupled to each other, so that the imaging sources 114 move together. As a more specific example, each imaging source 114 can be coupled to the gantry 118 of the imaging system 106 at different positions along the gantry 118 (e.g., different rotational positions along the gantry 118). In this way, as the gantry 118 moves (e.g., rotates) each imaging source 114 moves about the patient support 120 (and thus the patient).

In some non-limiting examples, the imaging detector 116 can be configured to receive and sense radiation imaging beams that are emitted from the imaging source(s) 114 and pass through the patient (and in some cases the patient support 120). The imaging detector 116 can be aligned with each of the imaging sources 114. For example, in the case of the imaging system 106 having one imaging source 114 (e.g., operating at different energy levels), the imaging detector 116 can be coupled to and can be aligned with the imaging source 114, so that as imaging source 114 moves, the imaging detector 116 moves with the imaging source 114. In this case, for example, the imaging detector 116 can be configured to acquire imaging data at different energy levels. For example, the imaging detector 116 can acquire imaging data from the sensing of radiation from a first radiation imaging beam having a first energy level (e.g., in which the first energy level is part of a first radiation energy spectrum of the first radiation imaging beam), and can acquire imaging data from the sensing of radiation from a second radiation imaging beam having a second energy level different from the first energy level (e.g., in which the second energy level is part of a second radiation energy spectrum of the second radiation beam that is different than the first energy spectrum). In some cases, the first radiation energy spectrum (e.g., a first x-ray photon energy spectra) of the first radiation imaging beam can overlap partially or can be completely separate from the second radiation energy spectrum (e.g., a second x-ray photon energy spectrum). In some configurations, the first energy level can be a local peak, a global peak, etc., in the first radiation energy spectrum, and the second energy level can be a local peak, a global peak, etc., in the second radiation energy spectrum. In some cases, while two energies are described this description can pertain to the imaging source(s) 114 emitting other radiation imaging beams other than the first and second radiation imaging beams, each being at a different energy level.

In some cases, the imaging detector 116 can operate in at least two modes (e.g., switchable by a computing device, such as the computing device 112), with one mode of operation having the imaging detector 116 tuned to acquire imaging data at the first energy level, and with a second mode of operation having the imaging detector 116 tuned to acquire imaging data at the second energy level. In some cases, this tuning of the imaging detector 116 can be adjusting (e.g., increasing or decreasing) the gain of the imaging detector 116 (e.g., adjusting the amplification of signals received from each sensing element of the imaging detector 116).

In some non-limiting examples, such as when the imaging system 106 includes two imaging sources 114 each operating at different energy levels, the imaging detector 116 can be moved and brought into alignment with one imaging detector 116 for acquiring imaging data at a first energy level, and then after acquiring imaging data at the first energy level, the imaging detector 116 can be moved and brought into alignment with the second imaging detector 116 to acquire imaging data at the second energy level. In some configurations, rather than having a single imaging detector 116 the imaging system 106 can include multiple imaging detectors 116, with each imaging detector 116 being tuned to sense radiation at a different energy level. In other words, each imaging detector 116 can operate to sense radiation at different energy levels, with in some cases, the energy levels not overlapping with each other. As an example, a first imaging detector 116 can be tuned to sense radiation at one energy level, and a second imaging detector 116 can be tuned to sense radiation at a second energy level. In some cases, this tuning can be based on different materials of the imaging detector 116. For example, the first imaging detector 116 can have a different scintillating material than the scintillating material of the second imaging detector 116. In some non-limiting examples, such as with two imaging sources 114 and with two imaging detectors 116, one imaging source 114 can be coupled to and aligned with one imaging detector 116, and the other imaging source 114 can be coupled to and aligned with the other imaging detector 116. In this way, each pair of an imaging source 114 and corresponding imaging detector 116 move together about the patient (e.g., and the patient support 120).

In some non-limiting examples, each imaging detector 116 can be structured in different (or similar) ways. For example, each imaging detector 116 can be an X-ray detector, and each imaging detector 116 can be an energy integrating detector, or can be a photon counting detector. In some cases, each imaging detector 116 can have different layers, such as a scintillating layer, and a light conversion layer. The light conversion layer can include an array of sensing elements (e.g., a two-dimensional array “2D” array), with each sensing element sensing light (e.g., visible light) that interacts with the sensing element to generate a signal (e.g., an electric signal).

In some non-limiting examples, each imaging source 114 and each imaging detector 116 can be coupled to the gantry 118, which can be moveable so that when the gantry 118 moves, the imaging sources 114 and the imaging detectors 116 move with the gantry 118. The gantry 118 can be implemented in different ways. For example, the gantry 118 can include a cylinder gantry, a ring gantry, a C-arm gantry, etc. In some specific cases, such as when the imaging system 106 includes multiple imaging sources 114 and multiple imaging detectors 116, the gantry 118 can include two C-arms. In this case, the first C-arm can include a first imaging source 114 situated at one end of the first C-arm and a first imaging detector 116 situated at an opposite end of the first C-arm, while the second C-arm can include a second imaging source 114 situated at one end of the second C-arm and a second imaging detector 116 situated at an opposite end of the first C-arm.

In some non-limiting examples, the system 100 can include a portal imaging system 108 (e.g., a portal imaging device, which can include an electronic portal imaging device (“EPID”)) that can have an imaging detector 122. The portal imaging system 108 is configured to sense radiation emitted from the radiation source 104 of the radiotherapy system 102. For example, the radiation source 104 emits a radiation therapy beam 124 towards the patient supported by the patient support 120. After the radiation therapy beam 124 passes through the patient (and in some cases the patient support 120), the radiation therapy beam 124 interacts with the imaging detector 122 of the portal imaging system 108 and the imaging detector 122 can acquire imaging data from this interaction. In some configurations, the imaging detector 122 (and the portal imaging system 108) can be coupled to the patient support 120 (e.g., so that the imaging detector 122 is situated below the patient).

In some non-limiting examples, the imaging detector 122 is configured to sense and receive radiation therapy beams that are emitted from the radiation source 104 (e.g., the radiation therapy beam 124) and pass through the patient (and in some cases the patient support 120). Thus, the imaging detector 122 can be aligned with the radiation source 104. Similarly to the imaging detector 116, the imaging detector 122 can be an X-ray detector, and can include different layers. For example, the imaging detector 122 can include a radiation sensing layer, or multiple radiation sensing layers (e.g., four), with each radiation sensing layer stacked on top of one another. Each radiation sensing layer can include a light conversion layer that can include a 2D array of sensing elements, a scintillating layer (e.g., situated on top of the light conversion layer) such as gadolinium oxysulfide (“GOS”), and a copper layer (e.g., situated on top of the scintillating layer) or other metal layer. In some cases, the imaging detector 122 can have a layer (situated below the other layers) that is a backing layer that absorbs radiation (e.g., lead, tungsten, etc.). In some configurations, the imaging detector 122 can have a sensing area that is larger than the projection of the radiation therapy beam (from the radiation source 104) onto the imaging detector 122.

As shown in FIG. 1, the system 100 can include a patient support 120 that supports a patient during imaging (e.g., by the imaging system 106) and radiation therapy (e.g., by the radiotherapy system 102). The patient support 120 can be implemented in different ways. For example, the patient support 120 can be a bed, a chair, a table, etc., which is moveable to position the patient support 120 (and thus the patient) to different positions and orientations relative to the radiation source 104. In particular, the patient support 120 can be translatable, rotatable, etc. In some cases, and as illustrated, the patient positioning system 110 is coupled to the patient support 120 so that the patient positioning system 110 can direct movement of the patient support 120. For example, the patient positioning system 110 can be a robotic arm, which can include the patient support 120 coupled to a distal end thereof (e.g., the end effector of the robotic arm). In this case, a computing device (e.g., the computing device 112) can cause the robotic arm and thus the patient support 120 to move to different positions and orientations relative to the radiation source 104. Thus, the robotic arm can have a number of degrees of freedom (e.g., three, four, five, six, etc.) to realize the different desired positions and orientations of the patient support 120. In some cases, regardless of whether the patient positioning system 110 is implemented as a robot arm, the patient positioning system 110 can include actuators, motors, etc., to move (e.g., translate, rotate, etc.) the patient support 120.

The computing device 112 can be implemented in a variety of ways. For example, the computing device 112 can be implemented as one or more processor devices of known types (e.g., microcontrollers, field-programmable gate arrays, programmable logic controllers, logic gates, etc.), including as general or special purpose computers. In addition, the computing device 112 can also include other computing components, such as memory, inputs, other output devices, etc. (not shown). In this regard, the computing device 112 can be configured to implement some or all the steps of the processes described herein, as appropriate, which can be retrieved from memory. In some non-limiting examples, the computing device 112 can include multiple control devices (or modules) that can be integrated into a single component or arranged as multiple separate components.

In some non-limiting examples, the computing device 112 can include typical components used such as a processor, memory, a display, inputs (e.g., a keyboard, a mouse, a graphical user interface, a touch-screen display, etc.), communication devices, etc. In some cases, the computing device 112 can simply be implemented as a processor. The computing device 112 can communicate with other computing devices and systems. In some non-limiting examples, the computing device 112 can implement some or all of the processes described below.

As shown in FIG. 1, the computing device 112 can be in communication (e.g., bidirectional communication) with each of the components of the system 100, and thus can transmit instructions to (e.g., cause components to implement a task) and can receive data from some or all the components of the system 100. For example, the computing device 112 can cause the radiation source 104 to move to a different position, cause the radiation source 104 to emit a radiotherapy beam, cause the imaging system 106 to acquire imaging data of the patient, cause the portal imaging system 108 to acquire imaging data of the patient, cause the patient positioning system 110 to move the patient support 120 (and thus the patient) to a different position, etc.

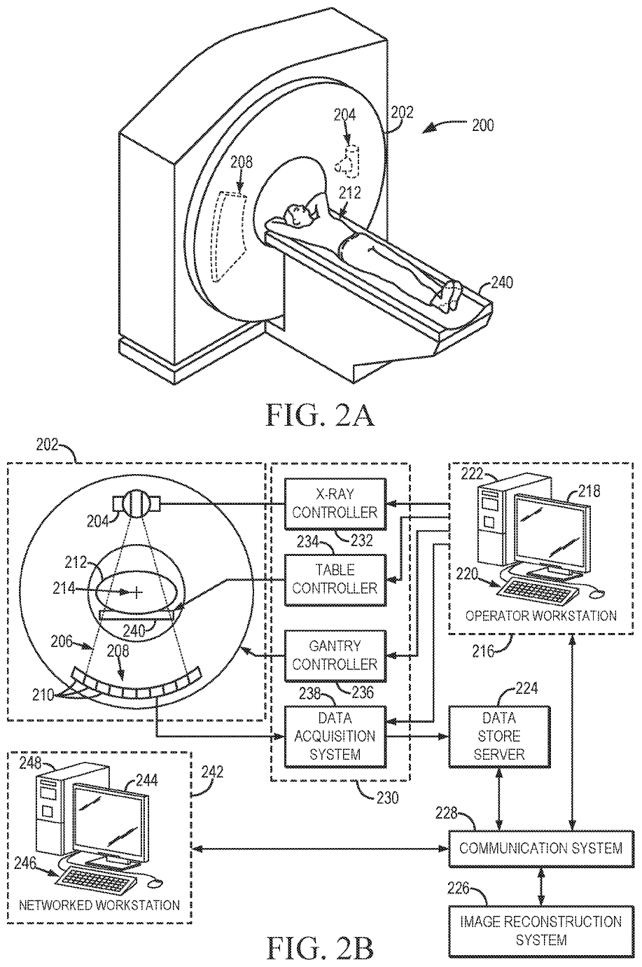

FIGS. 2A and 2B show an example of an x-ray computed tomography (“CT”) imaging system 200. The CT system includes a gantry 202, to which at least one x-ray source 204 is coupled. The x-ray source 204 projects an x-ray beam 206, which can be a fan-beam or a cone-beam of x-rays, towards a detector array 208 on the opposite side of the gantry 202. The detector array 208 includes a number of x-ray detector elements 210. Together, the x-ray detector elements 210 sense the projected x-rays 206 that pass through a patient 212, such as a medical patient or an object undergoing examination, that is positioned in the CT system 200. Each x-ray detector element 210 produces an electrical signal that can represent the intensity of an impinging x-ray beam and, hence, the attenuation of the beam as it passes through the patient 212. In some configurations, each x-ray detector 210 is capable of counting the number of x-ray photons that impinge upon the detector 210. During a scan to acquire x-ray projection data, the gantry 202 and the components mounted thereon rotate about a center of rotation 214 located within the CT system 200.

The CT system 200 also includes an operator workstation 216, which typically includes a display 218; one or more input devices 220, such as a keyboard and mouse; and a computer processor 222. The computer processor 222 can include a commercially available programmable machine running a commercially available operating system. The operator workstation 216 provides the operator interface that enables scanning control parameters to be entered into the CT system 200. In general, the operator workstation 216 is in communication with a data store server 224 and an image reconstruction system 226. By way of example, the operator workstation 216, data store server 224, and image reconstruction system 226 can be connected via a communication system 228, which can include any suitable network connection, whether wired, wireless, or a combination of both. As an example, the communication system 228 can include both proprietary or dedicated networks, as well as open networks, such as the internet.

The operator workstation 216 is also in communication with a control system 230 that controls operation of the CT system 200. The control system 230 generally includes an x-ray controller 232, a table controller 234, a gantry controller 236, and a data acquisition system 238. The x-ray controller 232 provides power and timing signals to the x-ray source 204 and the gantry controller 236 controls the rotational speed and position of the gantry 202. The table controller 234 controls a table 240 to position the patient 212 in the gantry 202 of the CT system 200.

The DAS 238 samples data from the detector elements 210 and converts the data to digital signals for subsequent processing. For instance, digitized x-ray data is communicated from the DAS 238 to the data store server 224. The image reconstruction system 226 then retrieves the x-ray data from the data store server 224 and reconstructs an image therefrom. The image reconstruction system 226 can include a commercially available computer processor, or can be a highly parallel computer architecture, such as a system that includes multiple-core processors and massively parallel, high-density computing devices. Optionally, image reconstruction can also be performed on the processor 222 in the operator workstation 216. Reconstructed images can then be communicated back to the data store server 224 for storage or to the operator workstation 216 to be displayed to the operator or clinician.

The CT system 200 can also include one or more networked workstations 242. By way of example, a networked workstation 242 can include a display 244; one or more input devices 246, such as a keyboard and mouse; and a processor 248. The networked workstation 242 can be located within the same facility as the operator workstation 216, or in a different facility, such as a different healthcare institution or clinic.

The networked workstation 242, whether within the same facility or in a different facility as the operator workstation 216, can gain remote access to the data store server 224 or the image reconstruction system 226 via the communication system 228. Accordingly, multiple networked workstations 242 can have access to the data store server 224 or image reconstruction system 226. In this manner, x-ray data, reconstructed images, or other data can be exchanged between the data store server 224, the image reconstruction system 226, and the networked workstations 242, such that the data or images can be remotely processed by a networked workstation 242. This data can be exchanged in any suitable format, such as in accordance with the transmission control protocol (“TCP”), the internet protocol (“IP”), or other known or suitable protocols.

The utilization of the CT imaging system 200 is only intended to be an example. The CT imaging system 200 shows, in one particular example, how imaging data can be obtained from a patient. However, in alternative non-limiting examples, other imaging modalities can be used. For example, imaging data used in the description below, can be derived from other radiation-based imaging systems.

In some non-limiting examples, the x-ray source 204 of the CT imaging system 200 can operate at different energies (e.g., to acquire one or more images at different energy levels, such as, for example, for a dual-energy imaging acquisition). For example, the x-ray source 204 can operate at a first energy level to emit a first radiation imaging beam (e.g., the x-ray beam 206) having a first energy at a patient (e.g., to acquire one or more images of the patient from the first radiation imaging beam passing through the patient to an x-ray detector, such as the detector array 208). As another example, the x-ray source 204 can operate at a second energy level to emit a second radiation imaging beam having a second energy different than the second energy (e.g., to acquire one or more images of the patient from the second radiation imaging beam passing through the patient to an x-ray detector, such as the detector array 208). In some cases, the x-ray source 204 can operate at a plurality of other energies (e.g., intermediate energies between the first energy level and the second energy level, other energies above and below the first energy level, other energies above and below the second energy level, etc.). In some configurations, the computer processor 222 can switch the operation energy of the x-ray source 204 (e.g., to change the energy of the radiation imaging beam). In some non-limiting examples, while the CT imaging system 200 has been described has having one x-ray source 204, in other configurations, the CT imaging system 200 can include multiple x-ray sources 204, each operating at a different energy level (e.g., emitting radiation imaging beams having different energies). Correspondingly, the CT imaging system 200 can include multiple detector arrays 208, each of which can be tuned to a particular energy level.

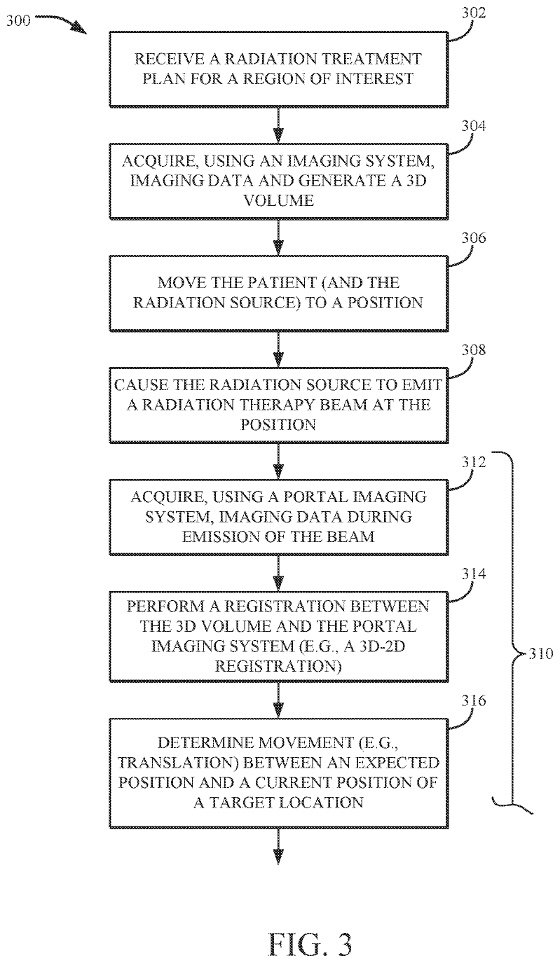

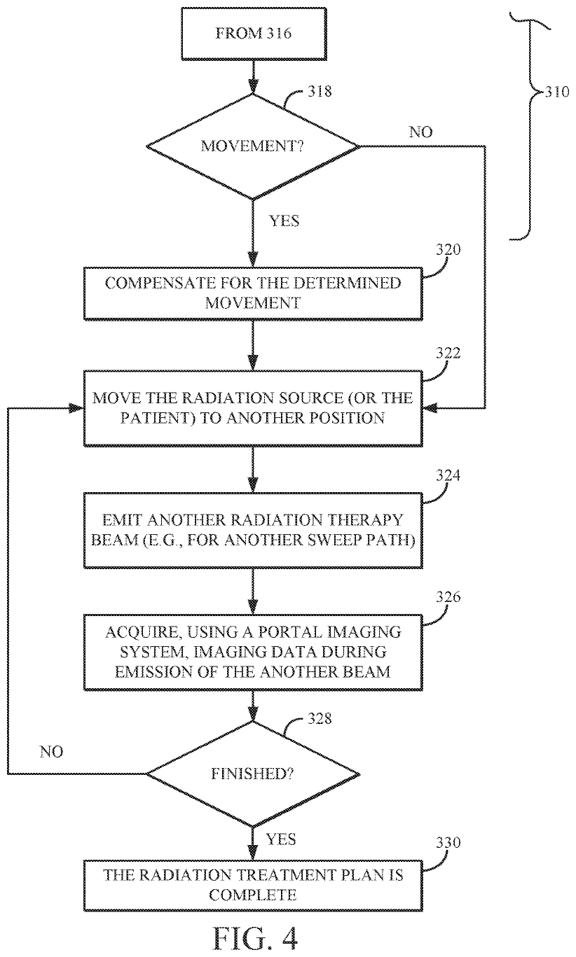

FIGS. 3 and 4 show a flowchart of a process 300 for delivering radiation therapy to a patient, which can include a process of tracking a patient (e.g., during radiotherapy). The process 300 can be implemented using the system 100. In addition, some or all of the blocks of the process 300 can be implemented using one or more computing devices (e.g., the computing device 112).

At 302, the process 300 can include a computing device receiving a radiation treatment plan for a region of interest of a patient. In some cases, the region of interest can include a target site (e.g., a tumor), one or more anatomical landmarks (e.g., for tracking), etc. In some configurations, the region of interest can be larger than the target site, one or more anatomical landmarks, etc. In some specific cases, the radiation treatment plan can be for a stereotactic radiation therapy to the target site (e.g., the spine). The radiation treatment plan can include a number of beam paths (e.g., a first beam path, a second beam path, etc.), each of which is defined from a radiation source and to the target site. In some cases, each beam path can define a corresponding energy that is to be used for the beam path, which can be different from the other beam paths. In addition, each beam path can define a corresponding beam duration (e.g., the amount of time a radiation therapy beam that aligns with the beam path is to be applied for), and a corresponding radiation dose (e.g., the maximum radiation dose that can be applied to the patient by a radiation therapy beam that aligns with the beam path).

In some cases, the radiation treatment plan can also include multiple sweep paths for the radiation source to follow (e.g., while the radiation therapy beam, corresponding to the sweep path, is being emitted), each of which can be different, and can include a respective corresponding beam path (e.g., which can be defined as the start of the corresponding sweep path). For example, a first sweep path can include a first beam path (e.g., being at the start of the sweep path, such as an end of the sweep path), a second sweep path (different from the first sweep path) can include a second beam path (different from the second beam path), and so on. Similarly to the beam paths, each sweep path can include a plurality of beam paths, each with different properties (e.g., energy levels, radiation doses, duration the radiation therapy beam is applied for while being emitted along the beam path, etc.). Thus, correspondingly, the properties of the radiation therapy beam can change along a sweep path (e.g., which can be defined by the radiation treatment plan).

In some cases, each sweep path can be continuous (e.g., the radiation therapy beam being emitted while following the sweep path), or non-continuous (e.g., the radiation therapy beam is not emitted while following portions of the sweep path). In addition, each sweep path can have different shapes. For example, some sweep paths can be curved (e.g., having different curvatures, lengths, distances from the patient, etc.), while other sweep paths can be non-curved. As a more specific example, the radiation treatment plan can include a first sweep path that is a first arc path (having different curvatures, lengths, distances from the patient, etc.), and a second sweep path that is a second arc path (different from the first arc path). In some non-limiting examples, the radiation treatment plan can include at least one standalone beam path (e.g., that are not swept), and at least one sweep path.

At 304, the process 300 can include a computing device acquiring, using an imaging system (e.g., the imaging system 106), imaging data. In some cases, the imaging data can include a first set of imaging data that has been acquired at a first energy level of the region of interest (or a different region of interest that includes the region of interest of block 302), and a second set of imaging data (different from the second set of imaging data) that has been acquired at a second energy level (different from the first energy level) of the region of interest (or a different region of interest that includes the region of interest of block 302). Thus, block 304 can include acquiring a first set of imaging data at a first energy level of a region of interest, and acquiring a second set of imaging data at a second energy level (different from the first energy level) of the region of interest (or other region of interest that at least partially overlaps with the region of interest of the first set of imaging data). In some non-limiting examples, the block 304 of the process 300 can also include, using the imaging system, acquiring a third set of imaging data at a different energy level (e.g., different from the first, and second energy levels), and acquiring a fourth set of imaging data at a different energy level (e.g., different from the first, second, and third energy levels), and so on. In this way, with multiple sets of imaging data can be acquired at different energy levels (e.g., with each different energy level being a peak energy of a respective radiation imaging beam delivered to the patient), the 3D volume of the patient can be better extrapolated to an energy level of a radiation therapy beam, which can facilitate better tracking of the patient (e.g., from patient movements). In some non-limiting examples, the energy level can be substantially 55 keV, 60 keV, 75 keV, 200 keV. In some cases, the energy level can be a high energy level, while the second energy level can be a low energy level, in which the high energy level is more than double the value of the low energy level. In some cases, the imaging data (including the first set of imaging data and the second set of imaging data) can include the target region as indicated in the radiation therapy plan.

In some non-limiting examples, acquiring the first set of imaging data can include causing one or more radiation sources of the imaging system (e.g., an X-ray source) to operate at a first energy level, and acquiring the second set of imaging data can include causing the one or more radiation sources of the imaging system to operate at a second energy level different from the first energy level. In some cases, the one or more radiation sources of the imaging system operating at the first energy level can include emitting a first radiation imaging beam towards a patient and the detector of the imaging system to acquire the first set of imaging data, in which the first radiation imaging beam can have a first energy, which can be part of a first radiation energy spectrum of the first radiation beam (e.g., in which the first energy can be a local peak, a global peak, etc., in the first radiation energy spectrum). Correspondingly, the one or more radiation sources of the imaging system operating at the second energy level can include emitting a second radiation imaging beam towards the patient and the detector of the imaging system to acquire the second set of imaging data, in which the second radiation imaging beam can have a second energy different than the first energy. In some cases, the second energy can be part of a second radiation energy spectrum of the second radiation beam (e.g., in which the second energy can be a local peak, a global peak, etc., in the second radiation energy spectrum).

In some cases, the first energy level (and the first energy) can be greater than the second energy level (and the second energy). For example, the first energy level (and the first energy) can be associated with the acquisition of one or more features of multiple different tissues types. In particular, the first energy level (and the first energy) can correspond to the attenuation of water for a first tissue type (e.g., in which the first tissue type includes a characteristic percentage of water as compared to surrounding tissue types, such as bone). For example, water can have a first K-edge (e.g., of less than 1 keV, such as less than about 541 eV such as for the K-edge of oxygen) and the first energy level (and the first energy) can be the same, larger, substantially larger, etc., than the first K-edge. In this way, the first set of imaging data can acquire features that correspond to water (e.g., at least because there is a significant spike in attenuation beyond, such as just beyond, the energy of the first K-edge). In some configurations, the first energy level (and the first energy) can be greater than (e.g., substantially greater than) the K-edge of a second tissue type (e.g., bone). In this way, features that correspond to the second tissue type (e.g., bone) do not obscure features from of the first tissue type (e.g., water based tissue types) due to, for example, the second tissue type attenuating much more than the first tissue type and thus obscuring the signal from the first type of tissue.

As another example, the second energy level (and the second energy) can be associated with the acquisition of one or more features of multiple different tissue types, or one tissue type (e.g., a single tissue type). In particular, the second energy level (and the second energy) can correspond to the attenuation of a second tissue type different from the first tissue type, which can be bone. For example, bone can have a second K-edge (e.g., of less than 4 keV, such as for the K-edge of calcium) and the second energy level (and the second energy) can be the same, larger, substantially larger, etc., than the second K-edge. In this way, the second set of imaging data can acquire features that correspond to bone (e.g., at least because there is a significant spike in attenuation beyond, such as just beyond, the energy of the second K-edge). In some cases, the second energy level (and the second energy) being less than the first energy level (and the first energy) can make features of the second tissue type more visible than features of the first tissue type (e.g., because the second tissue type attenuates much more than the first tissue type). In some configurations, the first energy level, the second energy level, and so on, can be greater than about 500 eV, greater than about 4 keV, etc.

In some configurations, the mass attenuation coefficient of the first tissue type (e.g., a water-based tissue type) at the first energy level (and the first energy, which can be about 55 keV) can be substantially the same as the mass attenuation coefficient of the first tissue type at the second energy level (and the second energy, which can be about 75 keV). Correspondingly, the mass attenuation coefficient of the second tissue type (e.g., bone) at the first energy level (and the first energy, which can be about 55 keV) can be different than the mass attenuation coefficient of the second tissue type at the second energy level (and the second energy, which can be about 75 keV) (e.g., the attenuation coefficient of the second tissue type can be higher at a higher energy level and vice versa). In this way, the intensity of a feature of at least the second tissue type (e.g., bone) can be different between a first image of (or constructed from) the first set of imaging data and a second image of (or constructed from) the second set of imaging data (e.g., because the attenuation is different at different energy levels). Accordingly, as described in more detail below, a combined image (or 3D volume) of the feature can allow for better tracking with the portal imaging system at least because the different images of the second tissue type can effectively extrapolate what the portal imaging system will see at higher energy levels. In other words, the difference between the intensities of the images that include the same feature (e.g., a bone) facilitates a predicable change in intensities so that larger attenuations from higher energy radiation beams that follow an extrapolated further change along the predicable change can track the same feature.

In some cases, each imaging data set can subject the patient to substantially the same amount of radiation during the image acquisition process (e.g., substantially 12 mGy, less than 12 mGy, etc.). In this way, even for higher energy acquisitions, the amount of radiation delivered can be minimized.

In some non-limiting examples, the block 304 of the process 300 can include a computing device generating a 3D volume of the region of interest of the patient (including the target region), using the first set of imaging data and the second set of imaging data. In some cases, this can include a computing device combining the first set of imaging data and the second set of imaging date to create a combined set of imaging data, and generating a 3D volume of a region of interest of the patient from the combined set of imaging data. For example, a computing device can construct a plurality of images from the first set of imaging data, and construct a plurality of images from the second set of imaging data. Then, a computing device can combine each pair of images (e.g., by superimposing the images, overlaying one image on another, etc.), with each image within each pair being from the same projection of the patient (e.g., an imaging detector and imaging source being at the same position), and with each pair having one image from the first set of imaging data and one image from the second set of imaging data. After, with each pair of images combined, a computing device can generate a 3D volume from the plurality of pairs of combined images.

In some non-limiting examples, a computing device can augment each image (e.g., from the first set of imaging data) before combining the image with another (e.g., from a second set of imaging data). For example, a computing device can filter each image, can segment out undesired portions of the image, can threshold each image, etc. In some cases, this can include a computing device for each image from the second set of imaging data, segmenting out undesired portion(s) of each image thereby resulting in an image of the second tissue type (e.g., bone). In some cases, this can result in an image of only the second tissue type.

In some non-limiting examples, the computing device can generate the 3D volume of the patient using multiple sets of imaging data acquired at different energy levels (e.g., two, three, four, etc.). In some cases, the computing device, using techniques from the field of multi-energy CT, generates a 3D CBCT image volume at a different energy level than the energy levels of the acquired sets of imaging data. For example, the different energy level can be greater than the energy level of each energy level of the acquired sets of imaging data. As another example, the different energy level can be substantially the same (or the same) as the energy level of a radiation therapy beam that is to be delivered to a patient according to the radiation treatment plan. In some cases, generating the 3D (CBCT) volume can include adjusting the pixel intensities of the first and second set of (CBCT) imaging data. For example, the first and second sets of imaging data can be used to mathematically model the pixel intensities that are to be seen at the different energy level (e.g., by linearly approximating pixel intensities relative to the energy level). For example, because the difference in energy level is known (e.g., between the first energy level and the second energy level), and the change in intensities is also known for the same tissue type (e.g., bone) including a feature thereof, the computing device can adjust the pixel intensities of the combined image or other image (e.g., by increasing the pixel intensities based on the energy level of a radiation therapy beam to be delivered to the patient such as the first radiation therapy beam of the block 308, which can include increasing the pixel intensities as a proportion of the energy level of the radiation therapy beam to be delivered to the patient). Correspondingly, in some cases, this can include adjusting (e.g., increasing) a voxel intensity of a 3D (CBCT) volume that was generated using the first and second sets of imaging data, based on the energy level of a radiation therapy beam to be delivered to a patient (e.g., by using the relationship between the change in voxel intensities and the change in energy between the first and second imaging sets to adjust the voxel intensities of the 3D volume to correspond to the different energy level of the radiation therapy beam).

In some non-limiting examples, the block 304 can include a computing device receiving a second 3D volume of the region of interest of the patient (e.g., by the computing device using an imaging system, such as the imaging system 106 to acquire imaging data and generate the second 3D volume from the patient), and comparing the 3D volume (e.g., generated at the block 304) with the second 3D volume. In some cases, this can include a computing device determining whether or not the patient has moved (e.g., relative to the patient support), based on the 3D volumes. For example, a computing device can register the 3D volume and the second 3D volume together to determine movements between the same anatomical features that are each within both 3D volumes. As a more specific example, a computing device can identify a first anatomical feature within the 3D volume (e.g., an organ, a bone structure, such as the spine, etc.), the computing device can identify the first anatomical feature within the second 3D volume, and determine movement (e.g., translation) between the first anatomical feature and the second anatomical feature. Then, in some cases, because the second 3D volume has already been related (e.g., registered) with the coordinate system of the radiotherapy system (e.g., the radiotherapy system 102) and in particular the radiation source of the radiotherapy system, the radiation source (or the patient support) can be moved to compensate for the determined movement.

At 306, the process 300 can include a computing device moving the patient (or the radiation source) to a position according to the radiation treatment plan. For example, this can include a computing device causing the radiation source to move to a first position that aligns with a first beam path, a first sweep path (e.g., an end of the sweep path), a first arc path (e.g., an end of the first arc path). In some cases, the first beam path, the first sweep path, or the first arc path can correspond to the first radiation therapy beam to be delivered to the patient. In some cases, this can include a computing device causing the radiation source, the patient support structure (e.g., via the patient positioning system), or both the radiation source and the patient support structure, to move to align the radiation source with a target region of the patient.

At 308, the process 300 can include causing the radiation source to emit a radiation therapy beam at the position (e.g., for a first sweep of the radiation source) according to the radiation treatment plan. In some configurations, the radiation therapy beam can have a first energy level, a first duration, a first orientation (e.g., angle with respect to the target site), and the radiation therapy beam can be a first radiation therapy beam of a plurality of different radiation therapy beams as defined by the radiation treatment plan (e.g., with each of the plurality of radiation therapy beams having one or more different characteristics including an energy level, a duration, an orientation, etc.). In some cases, this can include, with the radiation therapy beam being emitted towards the patient at the first position, sweeping the radiation source (and thus the radiation therapy beam) along a first sweep path (e.g., a first arc path), which can be implemented by rotating the gantry of the radiotherapy system (e.g., thereby rotating the radiation source about the patient). In some cases, as described above, this can include a computing device changing one or more properties of the radiation therapy beam (e.g., the duration, energy level, etc.) as the radiation source follows the first sweep path (or if the radiation source is maintained at the position).

At 310, the process 300 can include a computing device tracking a patient (e.g., that is undergoing radiation therapy), which can include blocks 312, 314, 316, 318 of the process 300. At 312, the process 300 can include a computing device acquiring, using a portal imaging system (e.g., an in particular a portal imaging device that can be an imaging detector), imaging data during emission of the radiation therapy beam. For example, the imaging data can be acquired throughout the entire treatment for the first sweep, or in other words, the imaging data can include subsets of imaging data (e.g., an image) each acquired from different positions of the radiation source (e.g., as it follows the path of the first sweep). Thus, the computing device can acquire, from the portal imaging system, a plurality of images (e.g., as many as a 100 images), with each image being of a different projection of the patient. In alternative configurations, such as if the position of the radiation source is maintained so that the radiation therapy beam emitted is not swept (e.g., the first beam path is a standalone beam path), the computing device can still acquire imaging data while the radiation therapy beam is being emitted, so that the imaging data includes a plurality of images acquired at different times (e.g., but of the same projection). In some cases, the imaging data (e.g., each image) acquired from the portal imaging system can be of a portion of the region of interest of the patient. In some cases, this portion can be an anatomical feature (e.g., a section of the spine, or other bone, etc.).

In some non-limiting examples, the imaging data (e.g., each image) acquired from the portal imaging system can be acquired at a third energy level. In some cases, the third energy level can be different than the first energy level and the second energy level. For example, the third energy level can be greater than the first energy level, and the second energy level. In some cases, the first energy level and the second energy level can be at a first order of magnitude (e.g., tens of KeV), and the third energy level can be at an order of magnitude larger than the first order of magnitude (e.g., hundreds of KeV).

In some non-limiting examples, such as when the imaging detector of the portal imaging system has more than one sensing layer (e.g., four sensing layers), imaging data from each sensing layer can be combined (e.g., averaged) for each acquired image (e.g., for each position of the radiation source). For example, at one point in time (e.g., that corresponds to the same position of the radiation source), imaging data (e.g., an image) from each sensing layer of the imaging detector can be combined to generate a single image for that point in time. In this way, each combined image (or composite image) using the imaging detector of the portal imaging system can have better image qualities (e.g., resolution), which can provide better tracking of the patient.

At 314, the process 300 can include a computing device performing a registration between the 3D volume (e.g., generated at block 304), and the portal imaging system (e.g., an imaging detector of the portal imaging system). In other words, this can include a computing device performing a 3D-2D registration between the 3D volume and the portal imaging system. In some cases, this can include performing a 3D-2D registration between a plurality a images (or an image) acquired from the portal imaging system. The computing device can relate (e.g., register) the coordinate system of the 3D volume to that of the portal imaging system and therefore the 2D images acquired from the portal imaging system can be used to track the 3D movement of the patient.

The 3D-2D registration can include a computing device generating a plurality of digitally reconstructed radiographs (“DRRs”), with each DRR predicting a frame of the portal imaging measurements for a hypothetical position change in the patient. Through an iterative process, a sequence of hypothetical patient position changes can be tested and compared with the actual portal imager measurements with the aim of converging upon the true patient position change. The set of portal imager frames participating in the registration process can consist of the full set of frames from the treatment arc, or a subset thereof. The size of the frame subset can be either increased to improve registration accuracy or decreased to mitigate computation time.

In some non-limiting examples, a mono-modal 3D-2D registration can be used, such as when the 3D CBCT image is derived, using dual energy CT techniques, to be at the same (or substantially the same) energy as the radiation therapy source (e.g., the x-ray source energy) of the 2D portal images. In other cases, a multi-modality registration (e.g., a multi-modality registration algorithm) can be used instead.

At 316, the process 300 can include a computing device determining movement of the patient based on the imaging data acquired from the imaging system and the imaging data acquired from the portal imaging system. For example, this can include a computing device determining movement (e.g., translation) between an expected position and a current position of a target location of the patient, based on the imaging data acquired from the imaging system (e.g., the 3D volume) and the portal imaging system. In some cases, this can include a computing device, using the 3D-2D registration (e.g., between the 3D volume and the portal imaging system) to determine movement between the expected position and the current position of a target location. In some cases, the target location can be the target site (e.g., the tumor of the patient), in other cases, the target location can be an anatomical feature (e.g., having a 3D shape) that appears in the imaging data from the imaging system and the imaging data from the portal imaging system. For example, the anatomical feature can be a portion of a section of the skeletal system of the patient (e.g., a section of the spine), an organ, etc.

At 318, the process 300 can include a computing device determining whether or not the patient has moved substantially (e.g., relative to a patient support). For example, this can include a computing device determining whether or not the current location of the target has deviated from its original location beyond an acceptable distance threshold (e.g., 2 cm, 1 cm, etc.). Alternatively, the computing device can determine that the patient has not moved substantially based on the expected position of the target location of the patient being substantially the same (e.g., deviating by less than 2 cm, less than 1 cm, etc.). If at 318, the computing device determines that the patient has moved substantially (e.g., moved a distance that is greater than the distance threshold), the process 300 can proceed to block 320. If, however, at 318, the computing device determines that the patient has not moved substantially (e.g., the distance moved, which can be determined by the computing device, being less than the distance threshold), the process 300 can proceed to block 322.

At 320, the process 300 can include a computing device compensating for the determined movement. For example, this can include the computing device determining an adjustment to the radiation treatment plan to compensate for the determined movement. As a more specific example, this adjustment to the radiation treatment plan can include a computing device changing a property (e.g., the duration, dose, energy level, position, orientation, etc.) of the next radiation therapy beam to be emitted to the patient (or other radiation therapy beams according to the radiation treatment plan), changing an order of application of radiation treatment beams for the patient (e.g., bypassing the next radiation therapy beam to be emitted, if its beam path does not intersect with the target site), removing a beam path from the radiation treatment plan (e.g., including truncating off a portion of a sweep path, or an arc path, with beam path(s) that do not intersect the target site), etc. In other cases, this can include modifying an orientation of the next radiation therapy beam (e.g., a second radiation therapy beam) to compensate for the determined movement. For example, the second radiation therapy beam (e.g., the next radiation therapy beam in the radiation treatment plan or another radiation therapy beam in the radiation treatment plan) can have an orientation relative to the target site (e.g., that is predetermined and based on an expected position of the patient). Thus, a computing device can adjust the orientation of the radiation source (e.g., by moving the radiation source, including by moving a robot arm, by rotating a gantry coupled to the radiation source, etc.) to adjust the position of the second radiation therapy beam to be emitted by the radiation source to compensate for the movement of the patient. In some cases, this can include modifying a position of the next radiation therapy beam (e.g., the second radiation therapy beam) to be delivered to the patient to compensate for the determined movement (e.g., the difference between the expected position of the patient and the actual position of the patient, each of which can be relative to the patient support). In some cases, the second radiation therapy beam (e.g., the next radiation therapy beam in the radiation treatment plan or another radiation therapy beam in the radiation treatment plan) can define a distance between the radiation source and the target site (e.g., that is predetermined and based on an expected position of the patient). Thus, a computing device can adjust the position of the radiation source (e.g., by moving the radiation source) to adjust the distance between the radiation source and the target site that is defined by the second radiation therapy beam (e.g., to be emitted by the radiation source) to compensate for the movement of the patient.

In some non-limiting examples, the block 320 of the process 300 can include a computing device updating a position of the patient based on the movement of the patient (e.g., determined at the block 318). In some cases, this can include the computing device shifting the coordinate system of the radiation source (e.g., of the radiotherapy system) to compensate for the movement. For example, an origin, or other reference point that is defined in the radiation treatment plan and is defined for the patient (e.g., a 3D volume), which can be used to relate the coordinate system of the radiation source (and thus the beam paths) to the coordinate system of the patient (e.g., the 3D volume) can be shifted (e.g., translated) in various directions to compensate for the determined movement. In other words, the movement can be determined to have occurred along one axis, two axes (e.g., the two axes extending along a plane parallel with the imaging detector of the portal imaging system), or three axes, and thus the origin (or other reference point) can be shifted along each axis by the determined movement for each axis. In this way, the beam paths defined in the radiation treatment plan (e.g., the orientations with respect to the target site, the distance between the radiation source and the target site, etc.) do not have to be individually changed.

At 322, the process 300 can include a computing device causing the radiation source (or the patient support, or both) to move to another position corresponding to another beam path according to the radiation treatment plan. For example, the computing device can cause the radiation source (or the patient support, or both) to move to a second position (e.g., a second orientation) that corresponds to a second radiation therapy beam to be delivered to the patient. In some cases, this another position is the next position that corresponds to the next beam path to be followed by a second radiation therapy beam (e.g., and thus the radiation source), according to the radiation treatment plan. In some cases, this another position can be originally defined relative to a reference point in the radiation treatment plan, while in other cases, the another position can be updated (e.g., or changed) to compensate for the movement of the patient.

In some non-limiting examples, including when the tracking process relies on a mono-modality registration, a computing device can adjust the 3D volume (e.g., to compensate for the energy level of the second radiation therapy beam). For example, the energy of the second radiation therapy beam can be different than the energy of the first radiation therapy beam (e.g., delivered at the block 308). Thus, the intensities of the voxels of the 3D volume can be adjusted to better align with the voxels to be seen at the energy level of the second radiation therapy beam. In other words, if the energy level of the second radiation therapy beam is higher than the energy level of the first radiation therapy beam, then the computing device can increase the intensities of the voxels, based on the second energy level (and vice versa). In this way, the intensities seen by the portal imaging system can better correspond to the intensities of the 3D volume (e.g., because they are effectively at substantially the same energy level). In some cases, after the voxels of the 3D volume have been adjusted (e.g., increased, decreased, etc.) based on the second energy level of the second radiotherapy beam to be delivered to the patient, the process 300 can perform a registration again (e.g., a 3D-2D registration).

At 324, the process 300 can include a computing device causing the radiation source (at the another position) to emit another radiation therapy beam (e.g., for another sweep path), according to the radiation treatment plan. In some cases, this another beam path (and another position) can correspond to another treatment section of the radiation treatment plan. For example, a sweep path having a plurality of beam paths can include the another beam path. In this way, because the radiation therapy beam of the previous iteration (e.g., at the block 312) was used to compensate for movement, a different radiation imaging acquisition (e.g., a CBCT imaging acquisition) does not need to be performed to compensate for movement for this another treatment section of the radiation treatment plan. As such, the total duration of the radiation treatment plan is reduced, while simultaneously preventing the need for subjecting the patient to additional radiation. In addition, a sweep path can include movement of the radiation source about the patient, and so compensation before a sweep path in particular (e.g., as opposed to a standalone beam) can significantly minimize errors or deviations between the desired delivered radiation from the sweep path and the actual delivered radiation from the sweep path. In other words, during a sweep path (e.g., an arc path) errors can compound as the radiation source moves from a start position of the sweep path to the end position of the sweep path. Stated another way, the distance between the desired position of a radiation therapy beam and the actual position of the radiation therapy beam can increase as the radiation source is moved along the sweep path. Accordingly, compensating for movements for sweep paths in particular can be important (e.g., because errors can compound quickly).

At 326, the process 300 can include a computing device acquiring, using the portal imaging system (e.g., the imaging detector, such as a portal imaging detector), additional imaging data during the emission of the another radiation therapy beam, which can be similar to the block 312 of the process 300. In some cases, because additional data (e.g., a plurality of images) has been acquired (e.g., of different energy levels, of different projections corresponding to different features of the patient, etc.) a computing device can perform another 3D-2D registration between the 3D volume and imaging data acquired from the portal imaging system acquired during different treatment sections of the radiation treatment plan (e.g., imaging data acquired from the block 312, and imaging data acquired from the block 326). In some cases, this can include constructing additional DRRs (e.g., with each DRR corresponding to the BEV for the beam paths of this treatment section). In this way, the 3D-2D registration between the 3D volume and the portal imaging system can be further refined and can improve (e.g., provide better tracking) because additional portal imaging data has been acquired. In some cases, the block 326 can be similar to the block 312.

In some cases, at the block 326, the process 300 can also include processes similar to the blocks 316, 318, 320 of the process 300. For example, this can include a computing device determining moment of the patient (e.g., based on the refined 3D-2D registration), an amount of movement (e.g., relative to a threshold), and compensation of the movement including adjusting the radiation treatment plan to compensate for the determined movement.

At 328, the process 300 can include a computing device determining whether or not the radiation treatment plan has been completed. For example, if the computing device determines that all the beam paths (e.g., all the radiation therapy beams to be delivered to a patient) according to the radiation treatment plan (or a modified version thereof), the computing device can determine that the radiation treatment plan has been completed, and the process 300 can proceed to the block 330 with the radiation treatment plan being complete. Alternatively, if at 328, the computing device determines that additional beam paths (e.g., some radiation therapy beams to be delivered to the patient have not yet been delivered) are to be utilized (e.g., which have not been already used), such as for additional treatment sections of the radiation treatment plan, the process 300 can proceed back to the block 322 to move the radiation source (or the patient, or both), to another position (e.g., which has already been compensated for movement at the block 326).

In some cases, prior to proceeding back to block 322 because additional data (e.g., a plurality of images) has been acquired (e.g., of different energy levels, of different projections corresponding to different features of the patient, etc.) a computing device can perform another 3D-2D registration between the 3D volume and imaging data acquired from the portal imaging system acquired during different treatment sections of the radiation treatment plan (e.g., imaging data acquired from the block 312, and imaging data acquired from the block 326). In some cases, this can include constructing additional DRRs (e.g., with each DRR corresponding to the BEV for the beam paths of this treatment section). In this way, the 3D-2D registration between the 3D volume and the portal imaging system can be updated to reflect new changes in the position of the patient anatomy during the most recent treatment delivery.

### Examples

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

Some non-limiting examples of the disclosure provide 3D anatomy localization during volumetric modulated arc therapy (“VMAT”) delivery. In some cases, the radiation treatment beam can be used as a source for tracking the patient. For example, a portal imaging system can acquire 2D images during a first treatment arc of the radiotherapy session. Then, a 3D-2D registration can be performed between a 3D volume (e.g., acquired from a different imaging acquisition such as a CBCT imaging acquisition) and the portal imaging system (e.g., the 2D images acquired from the portal imaging system). In this way, motion can be detected, measured, and compensated, before the next treatment arc for the radiotherapy session. Accordingly, rather than a CBCT imaging acquisition, which requires additional radiation delivered to healthy tissues, the radiation treatment beam during a previous treatment arc can be used to quickly and accurately evaluate patient position by using the 3D to 2D registration.

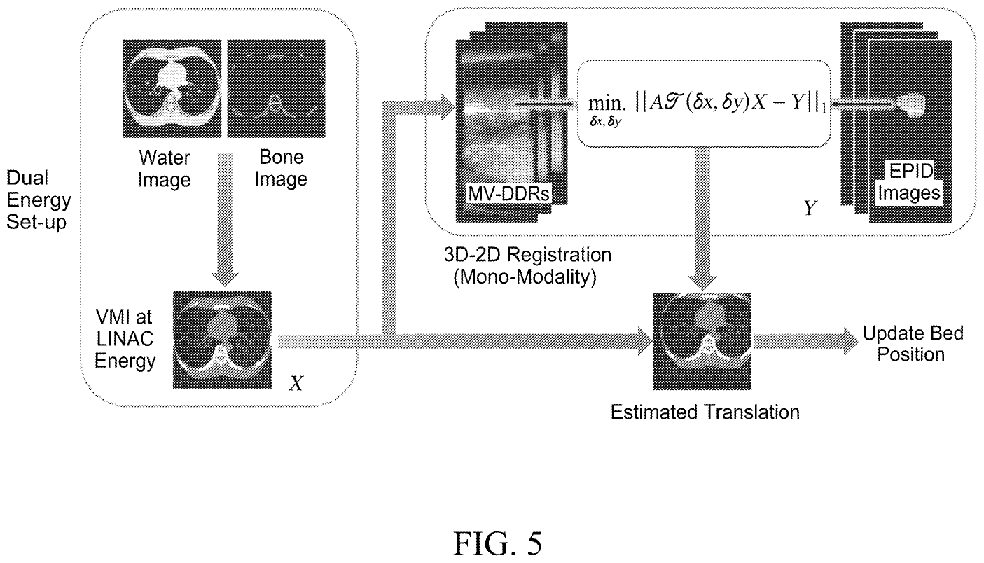

FIG. 5 shows a schematic illustration of a process that was used to track movement of digitally constructed images.

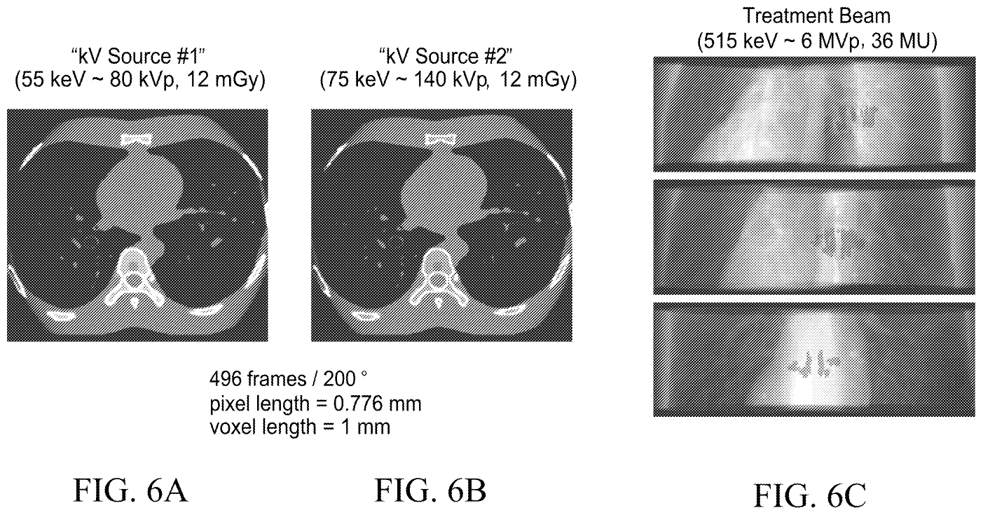

FIGS. 6A-6C show images from a digital thorax study. For example, FIG. 6A shows a digital thorax image that was generated according to a simulated first energy level, FIG. 6B shows a digital thorax image that was generated at a simulated second energy level, and FIG. 6C shows images from a simulated treatment beam showing tracking of the spine between images.

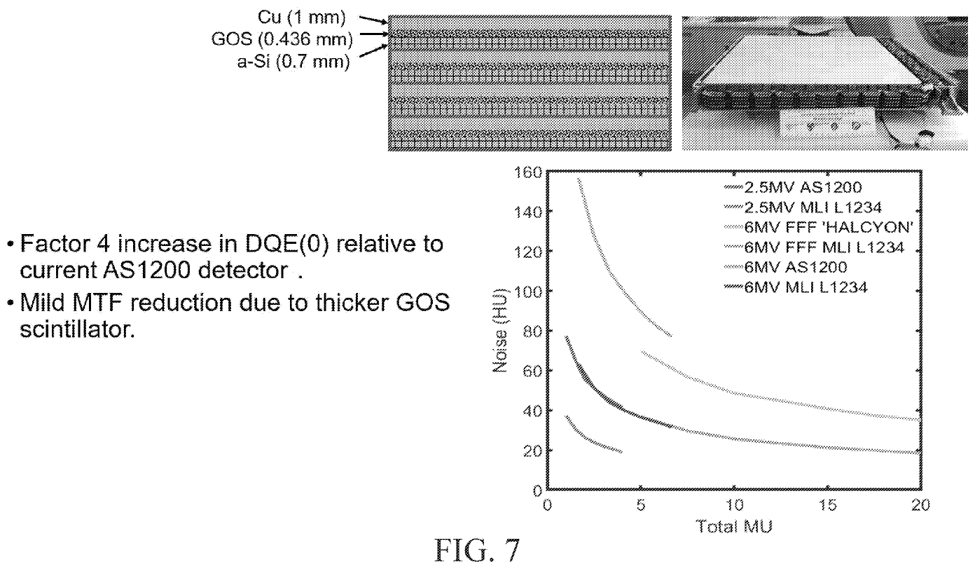

FIG. 7 shows an experimental setup of an imaging detector for a portal imaging system. As shown in FIG. 7, the portal imaging detector can have multiple sensing layers (e.g., illustrated as four sensing layers), with each sensing layer sensing radiation interacting therewith. For example, each sensing layer can have a light conversion layer (e.g., a-Si), a scintillating layer (e.g., GOS) positioned on top of the light conversion layer, and a copper conversion layer positioned on top of the scintillating layer. In some cases, with additional sensing layers (e.g., four) the effective quantum efficiency of the imaging detector can be increased by a factor (e.g., four factor increase).

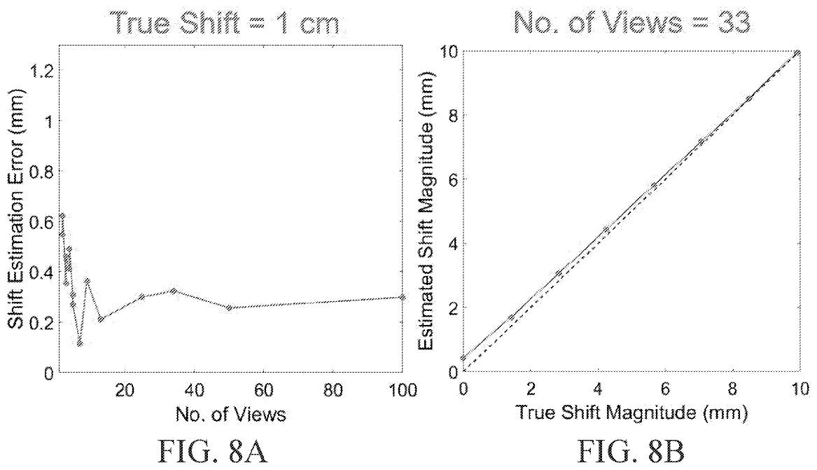

FIG. 8A shows a graph of the shift estimation error verses the number of views for a true shift in 1 cm for the digital thorax study. FIG. 8B shows a graph of the estimated shift magnitude verses true shift magnitudes for the digital thorax study.

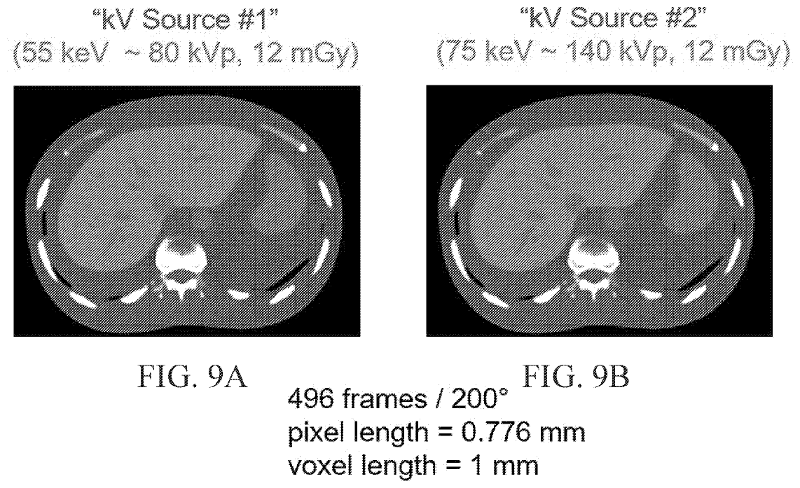

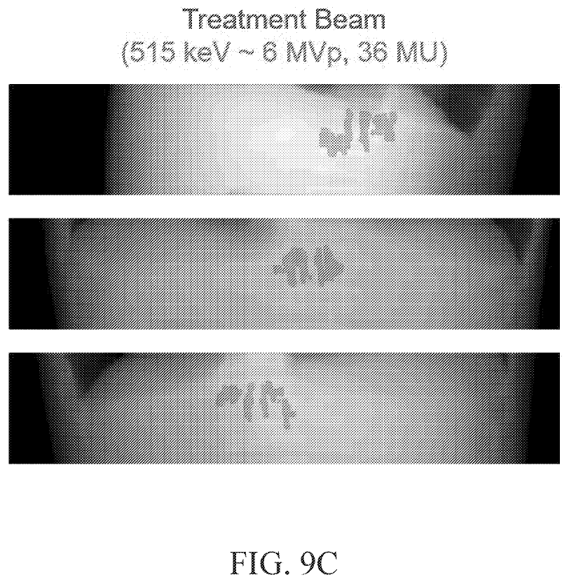

FIGS. 9A-9C show images from a digital abdomen study. For example, FIG. 9A shows a digital abdomen image that was generated according to a simulated first energy level, FIG. 9B shows a digital abdomen image that was generated at a simulated second energy level, and FIG. 9C shows images from a simulated treatment beam showing tracking of the spine between images.

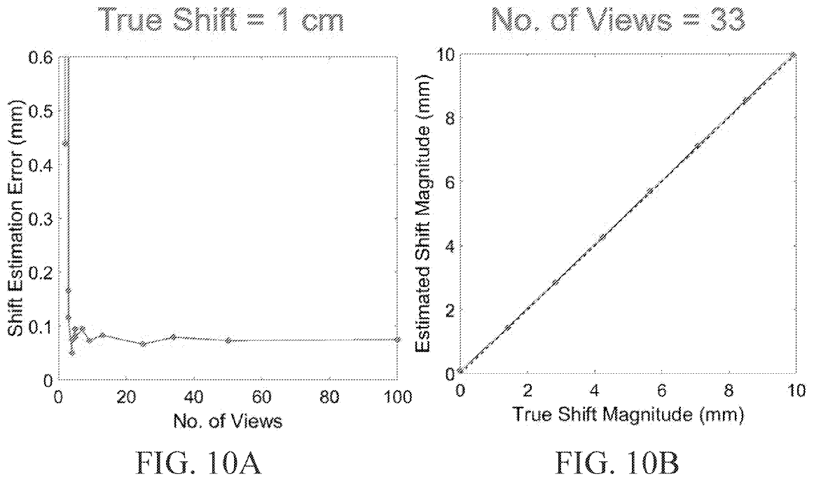

FIG. 10A shows a graph of the shift estimation error verses the number of views for a true shift in 1 cm for the digital abdomen study. FIG. 10B shows a graph of the estimated shift magnitude verses true shift magnitudes for the digital abdomen study.

As shown in at least the graphs, the tracking with the dual energy setup can achieve sub-millimeter positioning accuracy in preliminary tests. In addition, this tracking can reduce manual effort and repeated CBCT exposure in spine stereotactic radiation therapy (“SBRT”) or other radiation therapies, and may improve dose accumulation accuracy.

The present disclosure has described one or more preferred non-limiting examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the following description or illustrated in the following drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, ±5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

Various features and advantages of the disclosure are set forth in the following claims.

What is claimed is:

1. A system for delivering radiotherapy to a patient, the system comprising:
a radiotherapy system having a radiation source;
an imaging system;
a portal imaging system configured to sense radiation emitted from the radiation source of the radiotherapy system; and
a computing device configured to:
receive a radiation treatment plan for the patient, the radiation treatment plan defining a first beam path to a target site of the patient and a second beam path to the target site of the patient;
receive, from the imaging system, first imaging data, the first imaging data including a first set of CBCT imaging data acquired at a first energy level, and a second set of CBCT imaging data acquired at a second energy level different from the first energy level, the first and second sets of imaging data each including a region of interest of the patient that includes the target site;
cause the radiation source to emit a radiation therapy beam along the first beam path to the target site of the patient;
receive, from the portal imaging system, x-ray imaging data acquired during emission of the radiation therapy beam along the first beam path;
determine movement of the patient based on the CBCT and portal imager data; and
adjust the radiation treatment plan to compensate for the determined movement of the patient.

2. The system of claim 1, wherein the computing device is further configured to:
generate, using the first and second sets of CBCT imaging data, a three-dimensional (3D) volume of the region of interest of the patient at the energy of the radiation therapy beam;
receive or generate, using the x-ray imaging data, a plurality of BEV portal images;
perform a 3D-2D registration between the 3D volume and the portal imaging system using the plurality of BEV images; and
determine movement of the patient, based on the 3D-2D registration.

3. The system of claim 2, wherein the computing device is further configured to:
generate, using the 3D volume, a plurality of two-dimensional (2D) digital radiograph images; and
perform the 3D-2D registration using the 2D digital radiograph images and the plurality of BEV portal images.

4. The system of claim 2, wherein the 3D-2D registration is a mono-modality registration.

5. The system of claim 1, wherein the radiation treatment plan defines:
a first sweep path of the radiation source that includes the first beam path; and
a second sweep path of the radiation source that includes the second beam path, the first sweep being different than the second sweep.

6. The system of claim 5, wherein the first sweep path is a first arc path to be followed by the radiation source, and
wherein the second sweep path is a second arc path to be followed by the radiation source.

7. The system of claim 5, wherein the computing device is further configured to:
generate, using the first imaging data, a three-dimensional (3D) volume of the region of interest of the patient;
perform a 3D-2D registration between the 3D volume and the portal imaging system using the x-ray imaging data; and
determine movement of the patient, based on the 3D-2D registration.

8. The system of claim 7, wherein the x-ray imaging data is acquired by the portal imaging system during emission of another radiation therapy beam along the second sweep path.

9. The system of claim 1, wherein adjust the radiation treatment plan to compensate for the determined movement includes the computing device being further configured to:
change a property of a next radiation therapy beam to be directed at the patient, according to the radiation treatment plan and based on the determined movement, the next radiation therapy beam corresponding to the next beam path to be followed by the next radiation therapy beam;
change an order of application of any of the remaining radiation treatment beams to be delivered to the patient according to the radiation treatment plan and based on the determined movement, each beam path of the radiation treatment plan corresponding with each radiation treatment beam;
remove a radiation treatment beam that was to be directed at a patient along a beam path from the radiation treatment plan, based on the determined movement; or
move the coordinates of a reference point defined previously in the radiation treatment plan, based on the determined movement.

10. The system of claim 9, wherein the computing device is further configured to change the property of the next radiation therapy beam to be directed at the patient, according to the radiation treatment plan and based on the determined movement, the next radiation therapy beam corresponding to the next beam path to be followed by the next radiation therapy beam, and
wherein the property of the next radiation therapy beam includes at least one of:
a duration of application of the next radiation therapy beam;
a radiation dose provided by the next radiation therapy beam;
an energy level of the next radiation therapy beam;
a position of the beam path to be followed by the next radiation therapy beam; or
an orientation of the beam path to be followed by the next radiation therapy beam.

11. The system of claim 8, wherein determining an adjustment to the radiation treatment plan to compensate for the determined movement includes the computing device being further configured to move at least one of the patient or the radiation source to compensate for the determined movement.

12. The system of claim 1, wherein the first set of CBCT imaging data acquired at the first energy level includes the imaging system emitting a first radiation imaging beam having a first radiation energy spectrum towards the patient and a detector array of the imaging system, the first energy level being a peak in the first radiation energy spectrum; and
wherein the second set of CBCT imaging data acquired at the second energy level includes the imaging system emitting a second radiation imaging beam having a second radiation energy spectrum towards the patient and the detector array of the imaging system, the second energy level being a peak in the second radiation energy spectrum.

13. A computer-implemented method for tracking a patient, the method comprising:
receiving, using one or more computing devices, a radiation treatment plan, the radiation treatment plan defining a first beam path to a target site of the patient and a second beam path to the target site of the patient;
receiving, using the one or more computing devices, first imaging data including:
a first set of CBCT imaging data of a region of interest of the patient and acquired at a first energy level, the region of interest including the target site; and
a second set of CBCT imaging data of the region of interest and acquired at a second energy level different from the first energy level;
receiving, using the one or more computing devices, x-ray imaging data acquired from a portal imaging system during emission of a radiation therapy beam along the first beam path;
determining, using the one or more computing devices, movement of the patient based on the first imaging data and the portal imaging data; and
updating, using the one or more computing devices, a position of the patient, based on the movement of the patient.

14. The computer-implemented method of claim 13, further comprising moving, using the one or more computing devices, the patient or a radiation source to compensate for the determined movement of the patient.

15. The computer-implemented method of claim 13, wherein updating the position of the patient based on the movement of the patient includes:
changing, using the one or more computing devices, a property of a next radiation therapy beam to be directed at the patient, according to the radiation treatment plan and based on the determined movement, the next radiation therapy beam corresponding to the next beam path to be followed by the next radiation therapy beam;
changing, using the one or more computing devices, an order of application of any of the remaining radiation treatment beams to be delivered to the patient according to the radiation treatment plan and based on the determined movement, each beam path of the radiation treatment plan corresponding with each radiation treatment beam;
removing, using the one or more computing devices, a radiation treatment beam that was to be directed at a patient along a beam path from the radiation treatment plan, based on the determined movement; or
moving, using the one or more computing devices, the coordinates of a reference point defined previously in the radiation treatment plan, based on the determined movement.

16. The computer-implemented method of claim 13, further comprising:
generating, using the one or more computing devices and the CBCT imaging data, a three-dimensional (3D) volume of the region of interest of the patient;
performing, using the one or more computing devices and the second imaging data, a 3D-2D registration between the 3D volume and the portal imaging system; and
determining, using the one or more computing devices, movement of the patient, based on the 3D-2D registration.

17. A method for delivering radiation therapy to a patient, the method comprising:
receiving a radiation treatment plan for the patient;
receiving first imaging data, the first imaging data being a dual-energy imaging acquisition;
delivering a radiation therapy beam to a patient;
receiving, from a portal imaging system, second imaging data, the portal imaging system sensing the radiation therapy beam to acquire the second imaging data; and
compensating for movement of the patient, based on the first imaging data and the second imaging data.

18. The method of claim 17, further comprising:
generating, using the first imaging data, a three-dimensional (3D) volume of the region of interest of the patient;
performing, using the second imaging data, a 3D-2D registration between the 3D volume and the portal imaging system; and
determining movement of the patient, based on the 3D-2D registration.

19. A system for delivering radiotherapy to a patient, the system comprising:
a radiation therapy source configured to move to different positions about the patient;
an imaging system including one or more radiation imaging sources and one or more detector arrays, the one or more radiation imaging sources being configured to emit different radiation imaging beams having different energies towards the patient and the one or more detector arrays to acquire imaging data of the patient at different energies;
a portal imaging device configured to sense radiation emitted from the one or more radiation sources to receive imaging data therefrom; and
a computing device configured to:
receive, from the imaging system, first imaging data of a region of interest of the patient that includes a target site;
receive, from the imaging system, second imaging data of the region of interest of the patient;
generate a 3D volume of the region of interest of the patient using the first imaging data and the second imaging data;
cause the radiation therapy source to emit a first radiation therapy beam at the target site of the patient according to a radiation treatment plan;
receive, from the portal imaging device, one or more beam eye view (“BEV”) images from the interaction between the first radiation therapy beam and the portal imaging device, after the first radiation therapy beam has passed through the patient;
determine a movement of the patient, based on the 3D volume and the one or more BEV images; and
update or change the radiation treatment plan, based on the determined movement of the patient.

20. The system of claim 19, wherein the first imaging data is acquired with the one or more radiation sources of the imaging system operating at a first energy level; and
wherein the second imaging data is acquired with the one or more radiation sources of the imaging system operating at a second energy level different from the first energy level.

21. The system of claim 20, wherein the first energy level is a first peak energy of radiation emitted by the one or more radiation sources; and
wherein the second energy level is a second peak energy of radiation emitted by the one or more radiation sources; and
wherein the first peak energy is different than the second peak energy.

22. The system of claim 20, wherein the first energy level corresponds to a first tissue type being visible in the first imaging data; and
wherein the second energy level corresponds to a second tissue type different from the first tissue type being visible in the second imaging data.

23. The system of claim 22, wherein the first tissue type is a water-based tissue type, and the second tissue type is bone.

24. The system of claim 20, wherein receiving first imaging data of the region of interest includes:
causing the one or more radiation sources of the imaging system to emit a first radiation imaging beam towards the region of interest of the patient, the first radiation imaging beam having a first radiation energy spectrum, the first energy level being a peak in the first radiation energy spectrum;
receiving, from the one or more detector arrays of the imaging system, the first imaging data from the interaction between the first radiation imaging beam and the one or more detector arrays; and wherein receiving first imaging data of the region of interest includes:
causing the one or more radiation sources of the imaging system to emit a second radiation imaging beam towards the region of interest of the patient, the second radiation imaging beam having a second radiation energy spectrum, the second energy level being a peak in the second radiation energy spectrum; and
receiving, from the one or more detector arrays of the imaging system, the second imaging data from the interaction between the first radiation imaging beam and the one or more detector arrays.

25. The system of claim 19, wherein determining the movement of the patient based on the 3D volume and the one or more BEV images includes:
performing a 3D-2D registration between the 3D volume and the portal imaging device using the 3D volume and the one or more BEV images; and
determining the movement of the patient, based on the 3D-2D registration.

26. The system of claim 25, wherein performing the 3D-2D registration between the 3D volume and the portal imaging device includes:
generating, using the 3D volume, one or more two-dimensional (2D) digital radiograph images, each 2D digital radiograph image corresponding to a position of the radiation therapy source relative to the patient during emission of the first radiation therapy beam; and
perform the 3D-2D registration by using the one or more 2D digital radiograph images and the one or more BEV images.

27. The system of claim 19, wherein the portal imaging device includes a plurality of sensing layers; and
wherein each of the one or more BEV images is generated using the plurality of sensing layers.

28. The system of claim 19, wherein causing the radiation therapy source to emit the first radiation therapy beam at the target site of the patient according to the radiation treatment plan includes causing the radiation therapy source to emit the first radiation beam at the target site while moving the radiation therapy source along an arc.

29. The system of claim 19, wherein the computing device is further configured to:
after updating or changing the radiation treatment plan, moving the radiation therapy source to a position relative to the patient; and
with the radiation therapy source at least at the position, causing the radiation therapy source to emit a second radiation therapy beam at the target site of the patient according to the radiation treatment plan.

30. The system of claim 20, wherein the one or more BEV images are one or more first BEV images, and wherein the computing device is further configured to:
receive, from the portal imaging device, one or more second BEV images from the interaction between the second radiation therapy beam and the portal imaging device, after the second radiation therapy beam has passed through the patient;
determine a further movement of the patient, based on the 3D volume and the one or more second BEV images; and
further update or change the radiation treatment plan, based on the determined further movement of the patient.

31. The system of claim 30, further comprising:
after further updating or changing the radiation treatment plan, moving the radiation therapy source to another position relative to the patient; and
with the radiation therapy source at least at the another position, causing the radiation therapy source to emit a third radiation therapy beam at the target site of the patient according to the radiation treatment plan.

32. The system of claim 30, wherein the computing device is further configured to:
perform a first 3D-2D registration between the 3D volume and the portal imaging device using the 3D volume and the one or more first BEV images;
determine the movement of the patient, based on the first 3D-2D registration;
perform a second 3D-2D registration between the 3D volume and the portal imaging device using the 3D volume and the one or more second BEV images; and
determine the further movement of the patient, based on the second 3D-2D registration.

33. The system of claim 19, wherein updating or changing the radiation treatment plan includes:
adjusting a reference point of the radiation treatment plan, based on the determined movement, wherein each radiation therapy beam of the radiation treatment plan is defined relative to the reference point; or
adjusting one or more other radiation therapy beams of the radiation treatment plan other than the first radiation therapy beam.

34. A method of irradiating a patient according to a radiation treatment plan, the method comprising:
receiving a 3D volume of a region of interest of the patient that includes a target site, the 3D volume having been generated by irradiating the patient with radiation imaging beams having different peak energies;
causing a radiation therapy source to emit a radiation therapy beam at the target site of the patient according to the radiation treatment plan;
receiving, from a portal imaging device, one or more beam eye view (“BEV”) images from the interaction between the radiation therapy beam and the portal imaging device, after the radiation therapy beam has passed through the patient;
determining a movement of the patient, based on the 3D volume and the one or more BEV images; and
compensating for the movement of the patient to more accurately deliver one or more additional radiation therapy beams to the target site of the region of interest of the patient, the one or more additional radiation therapy beams being according to the radiation treatment plan.

35. The method of claim 34, wherein the radiation therapy source emits the radiation therapy beam while the radiation therapy source moves about the patient along a first sweep path; and further comprising:
generating, using the 3D volume, one or more two-dimensional (2D) digital radiograph images, each 2D digital radiograph image corresponding to one or more different positions of the radiation therapy source relative to the patient during emission of the radiation therapy beam;
performing a 3D-2D registration between the 3D volume and the portal imaging device using the one or more 2D digital radiograph images and the one or more BEV images; and
determining the movement of the patient, based on the 3D-2D registration.

36. The method of claim 35, wherein the radiation therapy beam is a first radiation therapy beam, and further comprising:
after compensating for the movement of the patient, moving the radiation therapy source to a position relative to the patient; and
with the radiation therapy source at least at the position, causing the radiation therapy source to emit a second radiation therapy beam at the target site of the patient according to the radiation treatment plan.

37. The method of claim 36, wherein the radiation therapy source emits the second radiation therapy beam while the radiation therapy source moves about the patient along a second sweep path different from the first sweep path.

38. The method of claim 37, wherein the first sweep path is a first arc about the patient; and
wherein the second sweep path is a second arc about the patient.
